(19) United States
(12) Patent Application Publication
(10) Pub. No.: US 2016/0206550 A1
(43) Pub. Date: Jul. 21, 2016

(54) STROMAL DELLS DERIVED CONDITIONED MEDIUM, METHOD OF OBTAINING SAID CONDITIONED MEDIUM COMPOSITIONS, FORMULATIONS AND APPLICATIONS THEREOF

(71) Applicant: STEMPEUTICS RESEARCH PVT. LTD., Karnataka, India,Bangalore (IN)

(72) Inventors: Sudha BALASUBRAMANIAN, Bangalore (IN); Swathi SUNDAR RAJ, Bangalore (IN); Charan THEJ, Bangalore (IN); Ramesh RAMCHANDRA BHONDE, Bangalore (IN); Raviraja NEELAVAR SEETHARAM, Bangalore (IN); Anish SEN MAJUMDAR, Bangalore (IN)

(73) Assignee: STEMPEUTICS RESEARCH PVT. LTD., Bangalore, Karnataka (IN)

(21) Appl. No.: 14/912,306

(22) PCT Filed: Jun. 18, 2014
(86) PCT No.: PCT/IB2014/062341

§ 371 Date: Feb. 16, 2016

(30) Foreign Application Priority Data
Aug. 29, 2013 (IN) 3867/CHE/2013

(51) Int. Cl. A61K 8/98; C12N 5/0775; A61Q 19/00; A61K 8/64; A61Q 19/08

(52) U.S. Cl. CPC A61K 8/981; A61K 8/64; A61Q 19/08; A61Q 19/00; C12N 5/0663; A61K 2800/5922; C12N 2502/1358

(57) ABSTRACT

The present disclosure relates to a conditioned medium (CM) enriched with bioactive factor, its composition and the method of producing the CM. The method of obtaining the desired quantity of bioactive factors in conditioned medium comprises of pooling bone marrow derived mesenchymal stromal/stem cells, culturing the pooled cell and subjecting the cell culture to a cell feeding schedule at specified confluency and collecting the potent conditioned medium rich in bioactive factors at specific passage/time period. The method further aims at maximizing the probability of generating a conditioned medium with reduced biological variability and comprising large number of bioactive factors having specific biological function/property. The disclosure also relates to composition and formulation of the conditioned medium and their use in cosmetic and therapeutic areas.

### TECHNICAL FIELD

The present disclosure relates to a method of obtaining conditioned medium (CM) rich in bioactive factors from pooled bone marrow derived mesenchymal stem cells (BM MSC). The instant method ensures a higher yield of desired growth factors and cytokines, by the use of cells pooled from multiple donors, which normalizes the inherent variability in the levels of the same secreted by cells from individual donors and by providing a cell feeding schedule at specified confluency. The disclosure further relates to the formulation of the conditioned medium for application in cosmetic and therapeutic areas.

### BACKGROUND

Mesenchymal stem cells have gained lot of interest in therapeutic/cosmetic field for various indications including skin conditions and wound healing. The mechanism through which MSCs participate in tissue repair is mainly through the secretion of trophic factors. MSCs provide the required microenvironment through the secretion of wide range of growth factors, cytokines and chemokines through their secretome. MSC secretome provides a new approach of treatment modalities in regenerative medicine and also in cosmetic applications. Growth factors, cytokines and chemokines serve as a tool for the cells to communicate and these molecules can be traced from the conditioned medium (CM) or spent medium harvested from cultured cells (Shohara et al., 2012). Most recently there has been lot of preclinical studies with the CM substitute for the various cell based therapies. (Walter et al., 2010). All these studies have given a tremendous hope for the secretome field.

Further, processes for obtaining conditioned medium rich in cytokines and growth factors from bone marrow derived mesenchymal stem cells, human embryonic stem cells, amnion derived multi potent cells etc and the use of the same in therapeutic applications related to wound healing, hair growth, scar reduction and other dermatological disorders are known in the art. However, there exists a need for producing conditioned medium containing a desired type and amount of cytokine/growth factor for a specific therapeutic application that requires a particular cytokine/growth factor or particular combination of cytokine/growth factors in optimum quantity. Therefore, the instant disclosure aims to address such a requirement currently lacking in the art.

### STATEMENT OF THE DISCLOSURE

Accordingly, the present disclosure relates to a method of preparing a conditioned medium comprising bioactive factors secreted by mesenchymal stromal cells, said method comprising acts of: a. culturing the mesenchymal cells in a cell culture medium followed by expanding and harvesting of the cells; and b. subjecting the harvested cells to a process of: a. fed batch activation; b. fed batch activation followed by complete medium change; or c. complete medium change; or any combination thereof, to obtain the said conditioned medium; a method of preparing a conditioned medium comprising bioactive factors secreted by mesenchymal cells, said method comprising acts of: a. culturing the mesenchymal cells in a cell culture medium followed by expanding and harvesting of the cells; and b. subjecting the harvested cells to a process of fed batch activation followed by complete medium change to obtain said conditioned medium; a conditioned medium comprising bioactive factors secreted by mesenchymal cells; a composition comprising conditioned medium along with pharmaceutically acceptable excipient and additive; and a method of managing skin related condition, said method comprising act of administering the conditioned medium or a formulation thereof, to a subject in need thereof.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1 shows the variation in the VEGF secretion between three donors and it's normalization after pooling and culturing.

FIG. 2 shows the levels of expression of various cytokines and growth factors in the conditioned medium from pooled bone marrow derived mesenchymal stromal/stem cells, over and above the cell culture medium. Multiplex analysis using ELISA and Luminex® based assays is used for the estimation.

FIG. 3 shows the comparison of bioactive factors secreted from multipotent mesenchymal stromal/stem cells cultured by 3 different processes. (a) VEGF; (b) PGE 2; (c) TGF-Beta-1 and (d) Angiopoietin-1.

FIG. 4 shows the levels of (a) VEGF, (b) TGF beta 1 and (c) PGE-2 in conditioned medium generated by process 2 from three different batches.

FIG. 5 (a) shows the enhanced bioactive factors retention in the conditioned medium as shown by the levels of VEGF after concentration by TFF technique using molecular cut-off >3 kDa and >1 kDa (b) show the VEGF functionality by transwell migration assay using human umbilical vein endothelial cells.

FIG. 6 shows the anti-ageing effects of CM upon human foreskin fibroblast cells (HFF) treated with Tert-butyl hydroperoxide (tbOH). (a) shows the restoration of collagen in the tbOH damaged cells treated with 10×CM at the dosage of 5%, 10% and 50% (b) shows the restoration of elastin levels in tbOH damaged HFF-1 cells treated with 10×CM at doses of, 10% and 50%, compared to their respective 10× control medium; (c) shows the restoration of hyaluronic acid (HA) levels in tbOH damaged HFF-1 cells treated with 10×CM at doses of 5% and 10%, compared to their respective 10× control medium; (d) shows the increase in cyclin B1 levels in tbOH damaged HFF-1 cells treated with 10×CM at doses of 5%, and 50%, compared to their respective 10× control medium.

FIG. 7 shows the evaluation of protective effects of pre-treatment of 10×CM against collagen degradation caused by oxidative damage in dermal fibroblast cells.

FIG. 8 relates to a graph representing a comparative analysis in the proliferation potential of HFF cells in 5 dilutions of the conditioned media with control medium FIG. 9a shows the significant fibroblast migration which is indicative of positive skin renewal effects upon the treatment of HFF-1 cells with 10×CM.

FIG. 9b shows the effect of 10× control and 10×CM on HFF-1 cells migration which is indicative of positive skin renewal effects. The data points are indicated as mean±standard error of triplicate values.

FIG. 10 shows the cytoprotective/viability restorative effects of 10×CM against oxidative stress induced by tert-butyl hydroperoxide in human dermal fibroblast cells. The data points are indicated as mean±standard error of triplicate values.

FIG. 11 shows the anti-wrinkling effects of CM upon human foreskin fibroblast cells (HFF-1 cells) subjected to UV radiation and treated with 10×CM at doses of 10% and 50% compared to their respective control medium. (a) shows the restoration of elastin levels; (b) shows the decrease in caspase 3 activation; (c) shows the extent of protection against DNA lesions.

FIG. 12 shows the evaluation of anti-apoptotic effects of 10×CM against oxidative stress induced apoptosis in dermal fibroblast cells.

FIG. 13 shows a graphical representation of mean corneometer reading indicative of skin hydration levels with all the formulations.

FIG. 14 shows a graphical representation of mean mexameter reading indicative of erythema index with all the formulations FIG. 15a shows photographic images of non-irradiated animals (control group-G1) on day 1 in which the skin surface was smooth and even.

FIG. 15b shows photographic images of untreated animals (control group-G1) on day 4, no visible macroscopic changes (redness, swelling, wrinkles) were observed.

FIG. 15c shows photographic images of untreated animals (control group-G1) on day 6, no visible macroscopic changes (redness, swelling, wrinkles) were observed.

FIG. 15d shows photographic images of untreated animals (control group-G1) on day 8, no visible macroscopic changes (redness, swelling, wrinkles) were observed.

FIG. 16a shows photographic images of UVB-irradiated animals (G2) on day 1 in which no visible macroscopic changes were observed.

FIG. 16b shows photographic images of UVB-irradiated animals (G2) on day 4 with noticeable induction of wrinkles and desquamation seen in animals of group that was UV-B exposed.

FIG. 16c shows photographic images of UVB-irradiated animals (G2) on day 6 with substantial wrinkles visible appearance of wrinkles, dullness, roughness and desquamation seen in animals of group that was UV-B exposed.

FIG. 16d shows photographic images of UVB-irradiated animals (G2) on day 8 with severe wrinkles and desquamation seen in animals of group that was UV-B exposed.

FIG. 17a shows photographic images of formulation 1 treated animals (G5) on day 1 in which no visible macroscopic changes were observed.

FIG. 17b shows photographic images of formulation 1 treated animals (G5) on day 4.

FIG. 17c shows photographic images of formulation 1 treated animals (G5) on day 6.

FIG. 17d shows photographic images of formulation 1 treated animals (G5) on day 8.

FIG. 18a shows photographic images of formulation 3 treated animals (G6) on day 1 in which no visible macroscopic changes were observed.

FIG. 18b shows photographic images of formulation 3 treated animals (G6) on day 4.

FIG. 18c shows photographic images of formulation 3 treated animals (G6) on day 6.

FIG. 18d shows photographic images of formulation 3 treated animals (-G6) on day 8.

FIGS. 19a and 19b show the comparison of the VEGF and TGF-β in the conditioned medium generated from pooled bone marrow and Wharton's jelly derived mesenchymal stem cells, respectively.

### DETAILED DESCRIPTION

The present disclosure relates to a method of preparing a conditioned medium comprising bioactive factors secreted by mesenchymal stromal cells, said method comprising acts of:
a. culturing the mesenchymal cells in a cell culture medium followed by expanding and harvesting of the cells; and
b. subjecting the harvested cells to a process of:
   a. fed batch activation;
   b. fed batch activation followed by complete medium change; or
   c. complete medium change; or any combination thereof,
      to obtain said conditioned medium.

In an embodiment of the present disclosure, the mesenchymal cells are mesenchymal stromal cells or mesenchymal stem cells or a combination thereof; and wherein the mesenchymal cells are bone marrow derived mesenchymal cells.

In another embodiment of the present disclosure, the mesenchymal cells are isolated from individual donors and pooled to obtain pooled mesenchymal cells.

In another embodiment of the present disclosure, the mesenchymal cells are seeded and expanded as passage 4 cultures at a seeding density of about 1000 cells/cm² to about 10000 cells/cm² preferably about 1000 cells/cm²;

In another embodiment of the present disclosure, the cells are expanded to a confluency of about 50% and subjected to media change and further cultured till about 80% to about 90% confluency.

In yet another embodiment of the present disclosure, the stem cells are further seeded and expanded as passage 5 cultures at a seeding density of about 1000 cells/cm² to about 10000 cells/cm² preferably about 1000 cells/cm² till about 45% to about 50% confluency.

In yet another embodiment of the present disclosure, the fed batch activation comprises acts of:
a. adding about 500 ml of cell culture media to the passage 5 cells expanded till about 45% to about 50% confluency; and
b. allowing culturing of the cells till about 80% to about 90% confluency and harvesting the conditioned medium.

In yet another embodiment of the present disclosure, the fed batch activation followed by change of cell culture medium comprises acts of:
a. adding about 500 ml of cell culture media to the passage 5 cells expanded till about 45% to about 50% confluency;
b. allowing culturing of the cells till about 65% to about 70% confluency and subjecting the cells to 2 L of complete change of cell culture media; and
c. allowing culturing of the cells till about 80% to about 90% confluency and harvesting the conditioned medium.

In still another embodiment of the present disclosure, the complete change of cell culture medium comprises acts of:
a. subjecting the passage 5 cells expanded till about 45% to about 50% confluency to about 1.5 L of complete change of cell culture media; and
b. allowing culturing of the cells till about 80% to about 90% confluency and harvesting the conditioned medium.

In still another embodiment of the present disclosure, the conditioned medium is concentrated and enriched by ultrafiltration or tangential flow filtration using molecular weight cut-offs of about 1 kDa and above or about 3 kDa and above; and wherein after the concentration, amino acid selected from a group comprising L-Arginine and L-Glutamic acid or a combination thereof is optionally added to the conditioned medium at a concentration ranging from about 50 mM to about 100 mM.

In still another embodiment of the present disclosure, the method optionally comprises pooling of the conditioned media obtained from each of the three processes.

In still another embodiment of the present disclosure, the cell culture medium comprises components selected from a group comprising Dulbecco's Modified Eagle Medium-Knock Out, Dulbecco's Modified Eagle Medium-F12, Dulbecco's Modified Eagle Medium-Low Glucose, fetal bovine serum, L-alanine-L-glutamine, penicillin and streptomycin or any combination thereof.

In still another embodiment of the present disclosure, the cell culture medium comprises basic fibroblast growth factor at a concentration of about 1 ng/mL to about 10 ng/mL, preferably about 2 ng/mL to enhance secretion of the bioactive factors.

In still another embodiment of the present disclosure, the conditioned medium obtained by fed batch activation is enriched for Ang-1; wherein the conditioned medium obtained by fed batch activation followed by change of cell culture medium is enriched for TGF-β; and wherein the conditioned medium obtained by change of cell culture medium is enriched for VEGF and PGE-2.

The present disclosure relates to a method of preparing a conditioned medium comprising bioactive factors secreted by mesenchymal cells, said method comprising acts of:
a. culturing the mesenchymal cells in a cell culture medium followed by expanding and harvesting of the cells; and
b. subjecting the harvested cells to a process of fed batch activation followed by complete medium change to obtain said conditioned medium.

In an embodiment of the present disclosure, the mesenchymal cells are mesenchymal stromal cells or mesenchymal stem cells or a combination thereof; and wherein the mesenchymal cells are bone marrow derived mesenchymal cells.

In another embodiment of the present disclosure, the mesenchymal cells are isolated from individual donors and pooled to obtain pooled mesenchymal cells.

In yet another embodiment of the present disclosure, the fed batch activation followed by—complete medium change comprise acts of:
a. adding about 500 ml of cell culture medium to the passage 5 cells expanded till about 45% to about 50% confluency;
b. allowing culturing of the cells till about 65% to about 70% confluency and subjecting the cells to complete medium change by adding 2 L of cell culture medium; and
c. allowing culturing of the cells till about 80% to about 90% confluency and harvesting the conditioned medium.

The present disclosure relates to a conditioned medium comprising bioactive factors secreted by mesenchymal cells. The bioactive factors comprises of growth factors, cytokines, chemokines, anti-oxidants and other factors that are known to function or mediate a biological process, wherein the biological process includes cell proliferation and cell migration.

In another embodiment of the present disclosure, the bioactive factors are selected from a group comprising VEGF, TGF-b, PGE-2, PDGF, GDNF, IGFBP, FGF, GCSF M-CSF, angiogenin, angiopoietin, KGF, FGF7, BMP6, IGF1, laminin, MMP1, MMP2, MMP9, TIMP1 and TIMP2, HGF, SDF1 and LIF, IL-10, IL-10 or any combination thereof; and wherein the conditioned medium, optionally contains amino acid selected from a group comprising L-Arginine and L-Glutamic acid or a combination thereof at a concentration ranging from about 50 mM to about 100 mM.

In another embodiment of the present disclosure, concentration of the VEGF ranges from about 2 to about 10 ng/mL.

In yet another embodiment of the present disclosure, concentration of the TGF-b ranges from about 1 to about 5 ng/mL.

In yet another embodiment of the present disclosure, the concentration of PGE-2 ranges from about 0.8 to about 2 ng/mL.

In yet another embodiment of the present disclosure, the concentration of Angiopoietin-1 ranges from about 10 to about 12 ng/mL.

In yet another embodiment of the present disclosure, the concentration of HGF ranges from about 20 to about 200 ng/ml.

In yet another embodiment of the present disclosure, the concentration of SDF1 ranges from about 0.4 to about 3 ng/ml.

In yet another embodiment of the present disclosure, the concentration of IL-10 ranges from about 10 to about 50 ng/ml.

The present disclosure relates to a composition comprising conditioned medium along with pharmaceutically acceptable excipient or cosmetically acceptable carriers.

In an embodiment of the present disclosure, the excipient is selected from a group comprising additive, carrier, granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material and spheronization agents or any combination thereof.

In another embodiment of the present disclosure, the excipient is selected from a group comprising Hydroxy ethyl cellulose, Hydroxy propyl cellulose, Hydroxy propyl methyl cellulose, Carbopol, EDTA, Methyl paraben, Propyl paraben, Deionised water, Glycerin, DL-Panthenol, D-Panthenol, Phenoxyethanol, Allantoin, PEG, Purified water, Xanthan gum, Sodium PCA, Gluconolactone, Sodium Benzoate, Sodium Hydroxide, Phenoxyethanol, Ethylhexylglycerin, Sodium Polyacrylate, Caprylic or Capric Triglyceride, Mineral oil, Tri (PPG-3 Myristyl ether) Citrate, SorbitanLaurate, Trideceth-6, PEG-75 Lanolin, Beta Glucan, Sodium Hyaluronate, Phosphatidylcholine, Cholesterol, Essential Oil, DL-α-Tocopherol acetate and Cyclodextrin or any combination thereof.

In yet another embodiment of the present disclosure, the composition is formulated into dosage forms selected from a group comprising, oily suspensions, hydrogel, nanogel, wet tissues, ointment, patch, gel, lotion, serum, emulsion, creams, spray, drops, or any combination thereof.

The present disclosure relates to a method of managing a skin related condition, said method comprising act of administering the conditioned medium or a formulation thereof, to a subject in need thereof.

In an embodiment of the present disclosure, mesenchymal stromal cells refer to multipotent mesenchymal stem cells and the said terms can be used interchangeably.

In an embodiment, the term ‘skin’ throughout this disclosure is used to mean skin as an organ as a whole, or any components or variants thereof, such as outer covering, outer coating, cutis, derma, dermis, epidermis, integument, peel, pelt, sheath and surface. Further the term ‘skin conditions’ includes cosmetic skin conditions such as wrinkles, frown lines, scarring, folds, sagging, age spots, uneven pigmentation, thinning, elasticity, scarring, skin roughness and dryness, open pores and other skin conditions,

In an embodiment of the present disclosure, ‘complete medium change’ refers to removal of spent media and feeding the cells with fresh cell culture medium.

In an embodiment of the present disclosure, the term multipotent stem cells includes mesenchymal stromal cells, mesenchymal stem cells derived from bone marrow, adipose tissue, Wharton's jelly dental pulp and other known sources.

In an embodiment of the present disclosure the term carriers includes pharmaceutical carrier, cosmetically acceptable carries and dermatologically acceptable carriers etc. The term carrier as referred in this application means an excipient or an vehicle, used for administration of the active ingredient (CM) topically to skin.

In an embodiment of the present disclosure, Conditioned medium (CM) or spent medium refers to the medium rich in various different types of biological active factor secreted by the stem cells, which are hereinafter collectively referred to as the bioactive factors. The term ‘bioactive factors’ comprises of biological active growth factors, cytokines, chemokines, anti-oxidants, small molecules, ECM and other factors that are known to function or mediate a biological process, for example cell proliferation, cell adhesion, differentiation, inflammatory response, cell migration and other biological functions etc., Conditioned medium is obtained from the cell culture collected at 80-90% confluency of the culture (10-14 days) which is the confluency at which the cells are in the mitotic phase and there is maximum production of bioactive factors.

The present disclosure provides an efficient method for enhancing the final availability of bioactive factors within the conditioned/spent media derived from bone marrow derived multipotent mesenchymal stem/stromal cells (BM-MSCs) and also to reduce any biological variability. The method involves pooling the said BM-MSCs from multiple donors to reduce the biological variability. The pooling of mesenchymal cells maximize the probability of generating—conditioned medium representing large number of bioactive factors with specific biological function/property and also to compensate biological factors not secreted/or secreted on lower levels by one of the selected individuals being pooled. The pooled cells with reduced biological variability are then cultured in cell culture medium and subjected to selective cell feeding schedule/media change processes at specified confluency to enhance the cell secretion and collection of bioactive factors by separating the conditioned medium from the cultured mesenchymal stromal cells.

In an embodiment the mesenchymal stromal cells are pooled to reduce the biological variability and overall heterogeneity general observed in CM obtained from individual donors. The pooling leads to producing bioactive factors which can be used to improve the skin health for cosmetic applications.

In another embodiment of the present disclosure, a conditioned medium produced in a serum and xeno-free conditions. The cell culture medium used for cell expansion also includes serum and xeno-free medium used for culturing and maintaining the stem cells.

In another embodiment the pooled MSCs are subjected to a specific feeding cycle at specific confluency to obtain maximum amount of CM rich in all the bioactive factors.

In yet another embodiment the CM rich in bioactive factor was collected and concentrated using known technology. The concentration process involves using a specific molecular weight cutoff to retain all the required bioactive factors which benefit the skin health.

In yet another embodiment the CM is purified and sterilized by generally known methods in the art.

In yet another embodiment the CM is formulated for topical administration to skin/for various cosmetic applications.

In yet another embodiment the CM is used for the improving the skin conditions such as wrinkles, frown lines, scarring, folds, sagging, age spots, uneven pigmentation, thinning, elasticity, scarring, skin roughness and dryness, open pores and other skin conditions,

In an embodiment of the present disclosure, the method of culturing and pooling the BM-MSCs from multiple donors is previously known and is briefly as follows:
a) Cryopreserved cells at passage 1, from individual donors, are thawed at 37° C. in a water bath.
b) The cells in the freezing medium are revived with 9 part cell culture medium in (1:9 ratio), after which the sample is centrifuged at about 1200 to 1500 rpm for 10-20 minutes at room temperature.
c) The supernatant is discarded, the pellet is resuspended with cell culture medium and the cells are counted.
d) The cells from all the donors are mixed in equal proportion, after which the cells are counted, the viability is checked and the viable cells are plated for passage 2 at a seeding density of about 1000 cells/cm² in cell culture vessel including and not limiting to culture flask, cell stack etc. The culture vessels are transferred to a 5% CO₂ incubator at about 37° C.
e) Pooled cells are further passaged to P3 and cryopreserved for further use.
f) The pooled MSCs obtained at passage 3 (P3) are thawed and expanded to further obtain the conditioned medium as explained in the below examples.

In an embodiment of the present disclosure, the technology of the instant application is further elaborated with the help of following examples and figures. However, the examples should not be construed to limit the scope of the disclosure.

### Example 1

The instant example compares the secretion of bioactive factors between individual and pooled bone marrow derived mesenchymal stem cells.

Analysis of Bioactive Factor Secretion of Cells from Individual Donors:

Human bone marrow was obtained from healthy donors at KMC Manipal hospital, Manipal, India. Human bone marrow derived mesenchymal stromal/stem cells isolated from multiple donors preferably from at least 2, or more preferably from three healthy donors are individually cultured using a cell culture medium composition as described in table 1 for five passages. The cell culture medium is not limited to the composition given in table 1 but can include any known cell culture medium including serum-free and xeno-free culture medium or a combination thereof known in the art. The cells are cultured in 25 cm² tissue culture flasks, complete change of cell culture medium is given every 3^{rd} day till cultures reach 80-90% confluency. The conditioned medium is collected from the confluent (day 9-10) cultures at passage 5 and preserved at −80° C. for further analysis.

TABLE 1
Cell culture medium composition
CELL CULTURE MEDIUM COMPOSITION

| | |
|---|---|
| Dulbecco's Modified Eagle Medium - Knock | About 85 to about 95% |
| out (DMEM-KO) or Dulbecco's Modified | preferably about 88.5% |
| Eagle Medium - F12 (DMEM-F12) or | |
| Dulbecco's Modified Eagle Medium - Low | |
| Glucose (DMEM-LG) or Dulbecco's Modified | |
| Eagle Medium - High Glucose (DMEM-HG) or | |
| any other basal medium or serum free medium | |
| or xeno free medium known in the art | |
| Fetal Bovine Serum (FBS) | About 5 to about 15% |
| | preferably about 10% |
| L-alanine-L-glutamine(GLUTAMAX) | About 0.5 to about 2% |
| | preferably about 1% |
| Penicillin Streptomycin(PENSTREP) | About 0.1 to about 1% |
| | preferably about 0.5% |
| Basic Fibroblast growth factor (bFGF) | About 1 to about |
| The basic fibroblast growth factor (bFGF) is an | 10 ng/mL preferably |
| optional component which enhances the | about 2 ng/mL |
| secretion of bioactive factors. | |

When conditioned medium from bone marrow derived mesenchymal stem/stromal cells (BM:MSC) obtained from three individual donors were analyzed for secreted growth factors (GF), considerable inherent variation in the levels of several GFs among the three donors were found as represented in the table 2. Some GFs were not represented in certain donor samples.

TABLE 2
Variation in Individual BM-MSC Samples

| Growth factor | BMMSC 1 | BMMSC 2 | BMMSC 3 |
|---|---|---|---|
| VEGF | 56.80 | 107.86 | 38.39 |
| TGF- b1 | 291.17 | nd | nd |
| PDGF- AA | 217.92 | 185.34 | 226.07 |
| PDGF R a | nd | 161.38 | 43.13 |
| GDNF | 89.49 | 18.81 | nd |
| IGFBP- 2 | 623.05 | 615.73 | 75.25 |
| IGFBP- 4 | 355.17 | 185.07 | 10.83 |
| IGFBP- 6 | 122.38 | 136.15 | nd |
| FGF-4 | nd | nd | nd |
| GCSF | nd | nd | nd |
| M-CSF | nd | nd | nd |
| M-CSF R | 9.87 | 0.65 | 5.50 |

*nd = not detected

Table 2 indicates the relative levels of the various growth factors in conditioned medium from individual BM:MSC samples, over and above the medium control. Values represent arbitrary fluorescence units.

Analysis of Bioactive Factor Secretion from Pooled Cells:

Human bone marrow derived mesenchymal stem cells isolated from at least 2, or preferably three healthy donors are pooled and cultured using the cell culture medium as described in table 1 for five passages. The cell culture medium is not limited to the composition given in table 1 but can include any known cell culture medium including serum-free and xeno-free culture medium or a combination thereof known in the art. The cells are cultured in 25 cm² tissue culture flasks, complete change of cell culture medium is given every 3^{rd} day till cultures reach 80-90% confluency. The conditioned medium is collected from the confluent (day 9-10) cultures at passage 5 and preserved at −80° C. for further analysis.

Alternatively, the method of culturing the pooled cells in a large scale involves expanding he pooled cells at passage 3 as passage 4 cultures in one cell stacks (CS-1) at a seeding density of 1000 cells/cm², subjected to a change of the medium at 50% confluency and further cultured till 80-90% confluency is achieved. The pooled cells are then harvested and expanded as passage 5 cultures at a seeding density of 1000 cells/cm² in ten cell stacks (CS-10) till 45-50% confluency is achieved. Thereafter 500 mL of cell culture medium is added to each cell stack and the conditioned medium is collected from the cell stacks at 80-90% confluency of the cultures.

The table 3 represents the bioactive factor rich conditioned medium from pooled culture derived from three donors, where significant levels of growth factors important in therapeutic processes for skin conditions are observed. This data suggests that pooling contributes to averaging out/compensates the inherent individual variability among donors and therefore increases the probability of representation of the majority of important GFs in the conditioned medium.

TABLE 3
Growth factor secretion in pooled BMMSC Samples

| | IMP |
|---|---|
| Growth Factor | (Arbit. Values) |
| FGF- 4 | 12.60 |
| GCSF | 31.63 |
| GDNF | 62.30 |
| IGFBP- 2 | 1401.61 |
| IGFBP- 4 | 241.10 |
| IGFBP- 6 | 1416.73 |
| M- CSF | 18.97 |
| M- CSF R | 27.40 |
| PDGF R a | 35.74 |
| PDGF- AA | 217.42 |
| TGF- b1 | 57.89 |
| TGF- b 3 | 39.11 |
| VEGF | 540.94 |

Table 3 indicates the relative levels of the various growth factors in conditioned medium from pooled BMMSC samples, over and above the medium control. Values represent arbitrary fluorescence units.

It is also important to note here that individual BM-MSC samples produce different amounts of various bioactive factors, and in some cases, the levels of certain bioactive factors are very less in a sample from a particular donor [for example IGFBP-4 in donor 3, when compared with donors 1 and/or 2]. Thus, there is always variability and heterogeneity amongst the levels of—bioactive factors when a single donor is taken into account. On the other hand, from Table 3, it is clear that there appears to be more uniformity in the levels of the bioactive factors. Although, it could be possible that the sample of cells derived after pooling of various cells from multiple donors are less in some bioactive factors there would also be cases where the levels of growth factors/cytokines are improved significantly from that of the individual samples, as the pooling would have made the levels of growth factors/cytokines more homogeneous. It further maximizes the probability of generating mesenchymal stromal/stem cells derived conditioned medium representing large number of bioactive factors and others molecules with specific biological function/property and also compensates for biological factor not secreted by one of the selected individual being pooled. Thus, the aforementioned observation establishes the importance of pooling for reduction of variability and heterogeneity and ensures that when a final product derived from the pooled cells is obtained for specific therapeutic and or cosmetic applications, the overall levels of bioactive factors—are maintained.

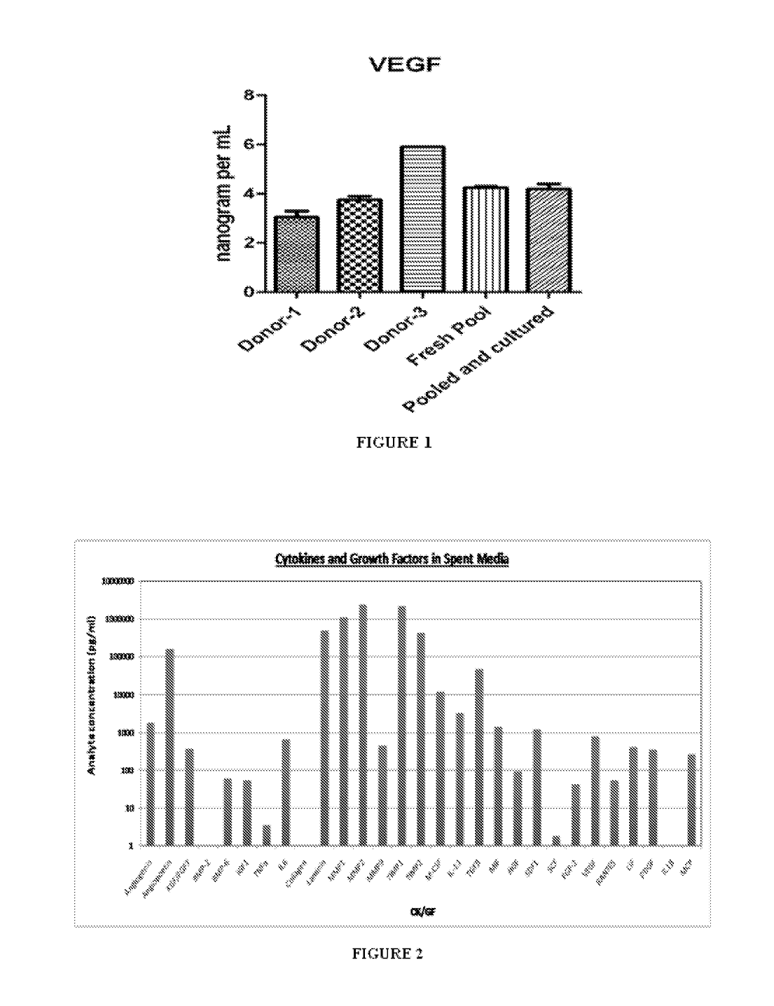

Further, FIG. 1 shows the variation in the VEGF secretion between three donors and it's normalization after pooling and culturing. FIG. 2 shows the levels of expression of various—bioactive factors in the conditioned medium from pooled bone marrow derived mesenchymal stem cells, over and above the cell culture medium.

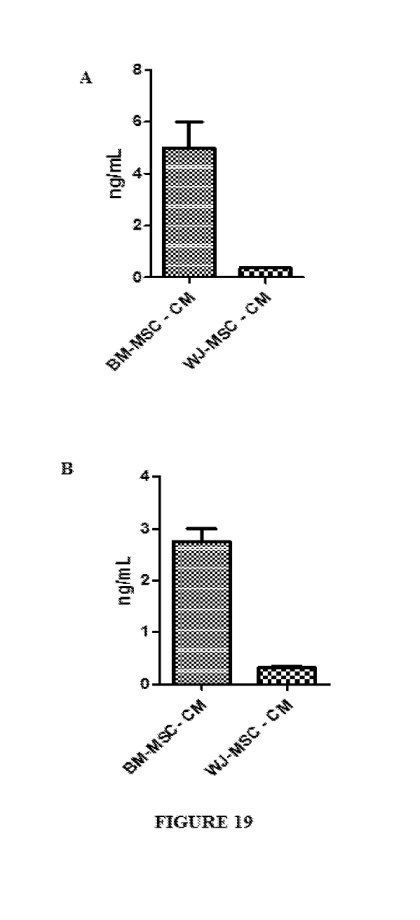

FIGS. 19a and 19b shows the comparison of the VEGF and TGFβ in the conditioned medium generated from pooled bone marrow and Wharton's jelly derived MSCs respectively. It is observed that the Wharton's jelly derived conditioned medium has less of both the factors when compared to the bone marrow derived conditioned medium.

### Example 2

### Process for Enhanced Production of Conditioned Medium

Initiation process: The cryopreserved pooled MSCs [obtained as per the process of isolation and pooling provided above] at passage 3 (P3) are thawed and expanded as P4 cultures in one Cell Stacks (CS-1) at a seeding density of 1000 cells/cm². A complete change of cell culture medium is given at around 50% of confluency (about 6-8 day cultures) and then cells are cultured until they become 80-90% confluent. Cells are then harvested and expanded as P5 cultures at a seeding density of 1000 cells/cm² in ten cell stacks (CS-10); and each CS-10 is filled with 1.5 L of the cell culture medium. One of the following 3 cell culture medium change processes (or also referred as feeding schedule processes) is performed once cells achieve 45-50% confluency (around day 7 cultures):

### Media Change Process 1:

In process 1, the medium change/cell feeding schedule comprises one partial medium change referred to as fed-batch activation or media top-up. In this fed-batch process fresh medium is added to the cell culture without removing the spent medium. The fed batch activation is performed with a volume of 500 ml per CS-10 on Day 06/07 (or when the cell are about 45-50% confluency) of culture to all the CS-10 cultures. Once the cultures reach 80-90% confluency, conditioned medium is collected and preserved from all CS-10.

### Media Change Process 2:

In process 2, the media change/cell feeding schedule comprises one partial and one complete medium change at a specific confluency. During the partial medium change, fed batch activation is performed with a volume of 500 mL per Ten Cell Stack (TCS) is on Day 06/07 (or when the cells reach about 50% confluency) of culture in all the CS-10. When the cells reach 65-70% confluency a complete medium change is carried out, wherein the spent medium is removed from the culture and fresh 2 L of cell culture medium is added per CS-10. Once the cultures reach 80-90% confluency, conditioned media is collected and preserved from all CS-10.

### Media Change Process 3:

In process 3, the media change/cell feeding schedule comprises only one complete medium change schedule. When the cells in TCS culture reach 45-50% confluency (or on days 6-8 of culture) the spent medium is removed completely from all TCS and replaced with 1.5 L/TCS of freshly prepared cell culture medium. Once the cultures reach 80-90% confluency, conditioned media is collected and preserved from all CS-10.

Approximately the conditioned medium production process for each of the three methods mentioned above requires about 10-15 days at the end of which the conditioned medium (CM) is collected for enrichment.

### Example 3

### Comparison of Bioactive Factors in the Conditioned Medium Generated from the Three Different Processes

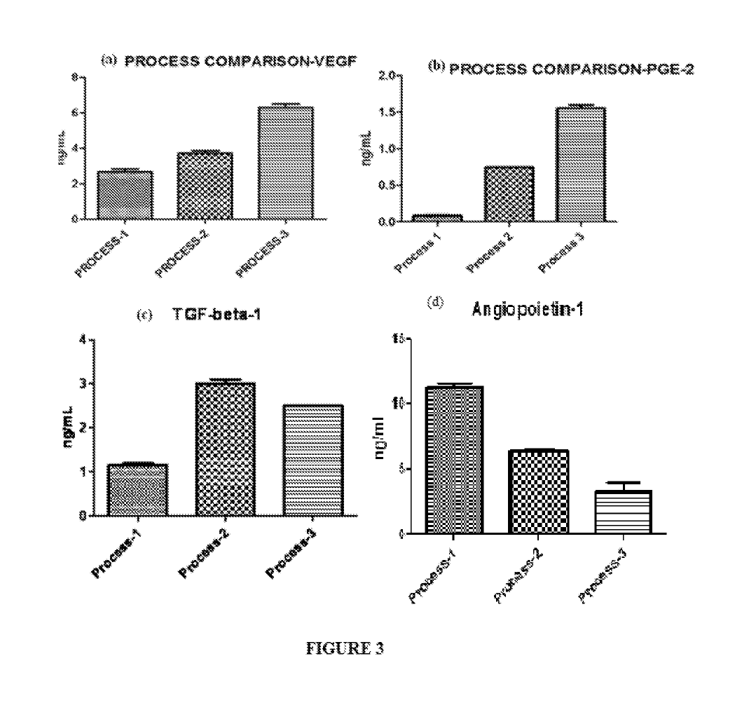

The three different processes (process 1, 2 and 3) are standardized for obtaining conditioned medium from pooled BM-MSC. Each individual process yields CM enriched for a specific set of therapeutic bioactive factors. FIGS. 3a, 3b and 3c compare the levels of VEGF, PGE-2 and TGF-beta-1 respectively, as obtained from the three different processes. For Example, CM enriched for Angiopoietin-1 can be obtained by following Process 1, CM enriched for TGF-beta can be obtained by following Process 2 and CM enriched for VEGF and PGE-2 can be obtained by following Process 3. A specific production processes can therefore be used to obtain tailor-made CM for a specific therapeutic application or set of applications demanding large amounts of the relevant GFs/Cytokines or other bioactive factors. E.g.: Process 2 can be employed for applications requiring cartilage regeneration, due to the large amounts of TGF-beta in the CM. Likewise, Process 3 can be employed for immune modulation due to the large amounts of PGE-2 in the CM. The secretion of specific bioactive factors in optimum levels during the processes 1, 2, and 3, by the pooled BM-MSCs is correlated to the rate of secretion, in vitro half-lives and stability of the growth factors/cytokines. Thus, different bioactive factors are secreted in the cell culture medium by the cells at different point of time, depending on the stage at which the conditioned medium is collected.

For example, owing to the long half life of Angiopoietin-1, whenever there is a requirement of Angiopoietin-1 for a specific therapeutic application, the pooled BM-MSCs are subjected to Process 1 that involves fed-batch activation on the 6^{th} or 7^{th} day of culturing wherein the cultures are 50% confluent, as a result of which the cell density increases and the secreted growth factor gets accumulated in the media. However, since VEGF is relatively secreted faster at desired confluency, whenever there is a necessity of VEGF, the pooled BM-MSCs are subjected to Process 3 which involves administering complete media change at 45-50% confluency on the 6^{th} to 8^{th} day of culturing, thereby inducing more production of the growth factor by the cells, which would ensure its optimum level in the media.

Thus, depending on the desired pharmaceutical or therapeutic application, specific conditioned medium can be arrived at, which comprises optimum levels of specific bioactive factors-, along with other growth factors/cytokines which are non-process specific.

Alternatively, CM from different individual processes can also be pooled selectively to obtain CM containing maximum quantities of relevant GFs/Cytokines for the given application. E.g. CM from process 1 & 3 can be pooled to obtain maximum enrichment of the angiogenic growth factors like Ang-1 and VEGF.

TABLE 4
Comparison of the bioactive factors in the CM generated
from three processes
Table 4 indicates the absolute levels of the growth factors and
cytokines in the CM generated from the three different processes.

| | Process - 1 | Process - 2 | Process - 3 |
|---|---|---|---|
| VEGF (ng/mL) | 2.6 ± 0.24 | 3.7 ± 0.36 | 6.3 ± 0.16 |
| TGF-b (ng/mL) | 1.15 ± 0.05 | 3.0 ± 0.1 | 2.4 ± 0.01 |
| PGE-2 (ng/mL) | 0.85 ± 0.005 | 0.7 ± 0.005 | 1.55 ± 0.05 |
| Angiopoietin- 1 | about 11.3 to | about 6.3 to | about 2.6 to |
| (ng/mL) | about 11.6 | about 6.4 | about 4 |

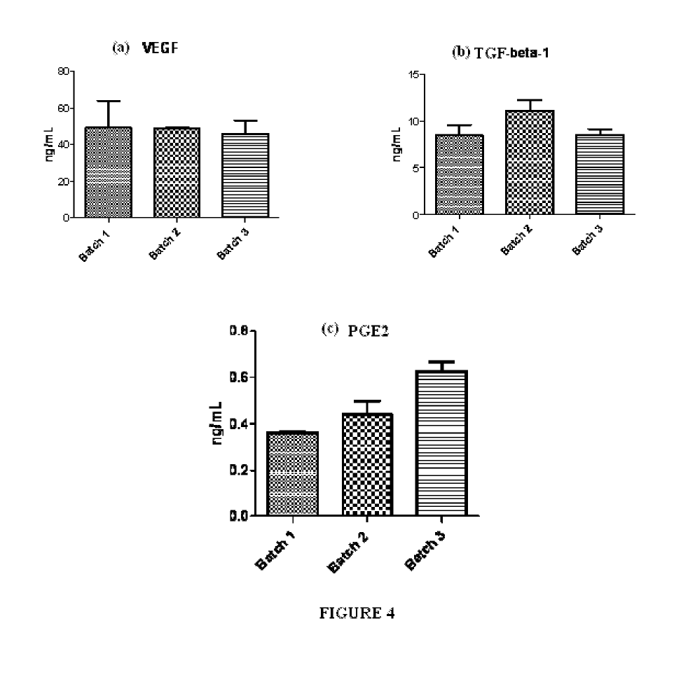

FIG. 4(a,b,c) provides the graph for VEGF, TGF beta and PGE-2 levels in the conditioned medium generated by process 2 from 3 from different batches. The batch comparison was performed after the TFF concentration, which resulted in a conditioned medium which is 10 times concentrated when compared to the original conditioned medium prior to the TFF. Thus the conditioned medium obtained is a 10× concentrate. The graph shows the consistency of process 2 generated conditioned medium from 3 different batches with the average concentration of 0.440±0.125 ng/ml and with variations which are statistically insignificant.

### Example 4

### Concentration of the Conditioned Medium

The CM collected from the above methods is optionally further concentrated and enriched by ultra-filtration/Tangential flow filtration (TFF) using defined molecular weight cut offs and the concentrated conditioned media was used for analytical estimation and formulation. The CM can also be concentrated by other known and established methods. In one of the embodiments, the concentration and enrichment by ultra-filtration/Tangential flow filtration (TFF) is performed by using molecular weight cutoffs of 3 KDa and above. While, in another embodiment the CM is concentrated using a molecular cutoff of 1 KDa and above. FIGS. 5a and 5b show the enhanced growth factor retention in the conditioned medium as shown by the levels of VEGF, after concentration by TFF concentration technique using molecular cut-off of 1 kDa, which is further proven to be functional by transwell migration assay using human umbilical vein endothelial cells (HUVEC) as described below. The migration of the HUVEC cells in response to the factors in CM that is concentrated using two different molecular cutoffs of 1 kDa and 3 kDa is compared to the migration of the HUVEC cells with response to the factors in Plain KO, KO+10% FBS and M199+20% FBS.

### Tangential Flow Filtration Technique:

Membrane-based Tangential Flow Filtration (TFF) unit operations are commonly used for clarifying, concentrating, and purifying proteins. In TFF, the fluid (feed) is pumped tangentially along the surface of the membrane. An applied pressure serves to force a portion of the fluid through the membrane to the filtrate side. In TFF equipment, a pump is used to generate pressure on the feed flow.

The TFF unit for the purpose of concentrating human bone marrow mesenchymal stem cell derived conditioned media (CM) utilizes a filter membrane with a cut-off of 1 kDa as this cut-off value is substantially lower than the molecular weight of the protein molecules that are desired to be retained. Particles which are of greater size are swept along with the tangential flow of the fluid. Thus, the tangential flow of the fluid prevents accumulation of particles on the flow path of the filtrate thereby enabling continuous operation at relatively high solids loads.

Diafiltration (DF) process can be performed in combination with the ultrafiltration run during protein concentration to enhance either product yield or purity. During DF (continuous diafiltration), buffer is introduced into the feed tank while filtrate is removed from the unit operation. This washes components out of the product pool into the filtrate, thereby exchanging buffers and reducing the concentration of undesirable species.

### TFF Run:

The membrane is installed and the TFF system is initialized (typically flushed with water and tested for water filtrate flow rate and integrity). About 1-2 litres of the CM is added to the feed tank) and the cross flow is established by maintaining the feed inlet pressure of 30 psi (P1) and a retentate pressure of 25 psi (P2). This run is continued till the CM is reduced to ten times its original volume (100-200 ml; depending on the feed quantity). The TFF concentration thus resulted in a conditioned medium which is 10 times concentrated when compared to the original conditioned medium prior to the TFF. Thus the conditioned medium obtained is a 10× concentrate. During this ultrafiltration cycle, permeate volume and changes in feed and retentate pressures are constantly recorded to note any change in flux. Once the ultrafiltration cycle is complete, diafiltration cycles are performed to enhance the purity of the final CM concentrate. In diafiltration process, Dulbecco's phosphate buffered saline is added into the feed tank in volumes equivalent to the CM retentate. Eight cycles of diafiltration are performed to obtain the final retentate with optimum purity.

After concentrating the conditioned media by TFF, the amino acids L-Arginine and L-Glutamic acid are added to the TFF concentrate, in their purified form solubilized in minimum quantity of Dulbecco's phosphate buffered saline, at a concentration of about 50 mM to about 100 mM. These amino acids function as protein stabilizers.

It is the 10× concentrate of the conditioned medium, which is further employed in the experiments and the formulations within the instant disclosure.

### Example 5

### Transwell Migration Assay

The Migration assay is performed using a transwell two-chamber cell culture method and Transwell inserts (3422; Corning, Cambridge, Mass.) with an 8 μm pore polycarbonate membrane. Human umbilical vein endothelial cells (HUVEC) are serum starved for 24 hours and 1×10⁵ cells/well are plated into the upper chamber of the transwell insert. In the lower chamber the cell culture media, plain media or conditioned media is added. 20 μg/mL of VEGF blocking antibody (R&D Systems-MAB293) is added as one of the condition. Cells are allowed to migrate for about 16-18 hours. After 18 hours of incubation at 37° C. in a humidified atmosphere containing 5% CO₂, the non-migrated cells are carefully removed using a pre-wetted cotton swab and the membrane is fixed with 4% paraformaldehyde and stained with haematoxylin for 10 minutes. After washing, the membrane is mounted with DPX and the cells that had migrated from the upper to the lower side of the membrane are counted under bright field microscope at 100× objective. (100× magnifications—Nikon 90i microscope). All the assays are done in triplicates. The data is represented as mean±SEM. The assay shows the concentrated conditioned medium is functional and the bioactive factors are not destroyed during the processing.

### Example 6

### Anti-Ageing and Anti-Wrinkling Effects of the Growth Factor/Cytokine Enriched Conditioned Medium from Pooled BM-MSCs

### In-Vitro Proof of Principle on the Anti-Ageing Effect of CM Generated by Process-1:

Ageing can be caused by both intrinsic and extrinsic factors including the body's biological clock and environmental threats such as free radicals and ultraviolet exposure that accelerates the skin's ageing process. Oxidative stress plays a central role in initiating and driving events that contribute to skin ageing at the cellular level by degrading vital building blocks of the skin including elastin and hyaluronic acid and even alters cellular renewal cycles.

Elastin protein fibres combine with collagen to give the skin elasticity while hyaluronic acid is found at the base of the dermis and forms a “cementing and gelling” base that binds to water molecules, allows nutrients and oxygen into the tissue and protects the dermal structural layer. In addition, cyclin B1 is a regulatory protein involved in mitosis whose levels are known to decrease in living cells during senescence. Thus, the protective effects/damage restorative effects against elastin and hyaluronic acid degradation and the anti-senescence effects of CM (at 10× concentration) of post-treatment of 10×CM against oxidative stress in dermal fibroblast cells are evaluated after 48 hours of treatment of cells exposed to Tert-butyl hydroperoxide (tbOH). For the purposes of these experiments, conditioned medium is obtained from culturing of MSCs derived from three donors, when pooled together and cultured by following Process 1.

Human foreskin fibroblast (HFF-1 ATCC® SCRC-1041™—source: human foreskin) cells are treated with 300 μM tbOH for 2 hours, followed by exposure of different doses of 10×CM and control medium for 48 hours. Elastin and hyaluronic acid levels are estimated in culture supernatant by ELISA method. For Cyclin B1, cells are lysed in lysis buffer to obtain cell lysates and protein levels are estimated by ELISA method.

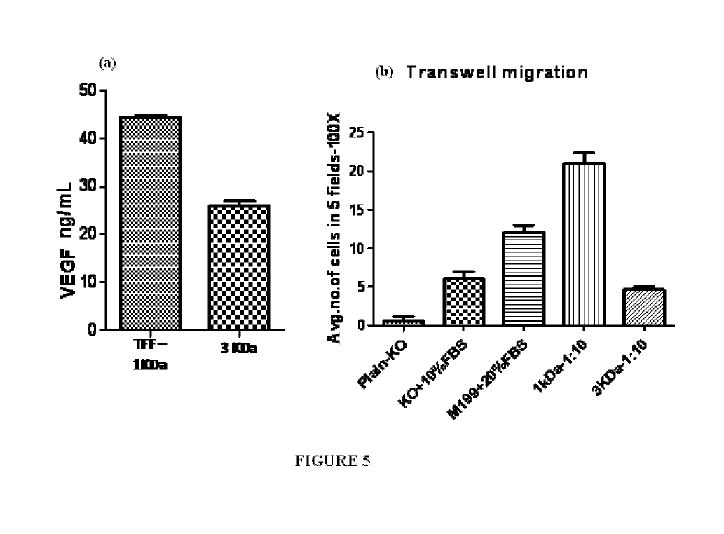

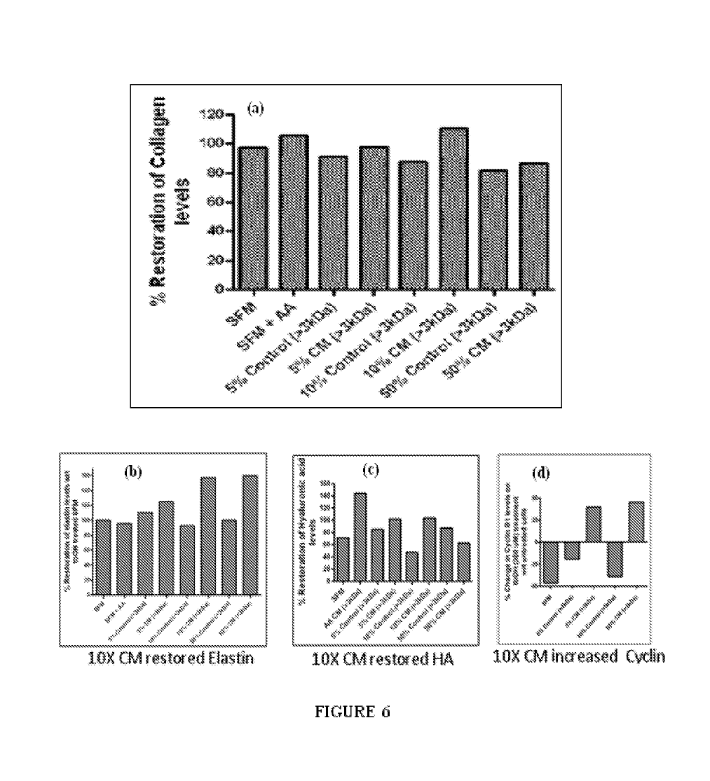

Post treatment of tbOH, damaged HFF-1 cells treated with 10×CM led to 7%, 23% and 6% restoration of collagen levels at the doses of 5%, 10% and 50% respectively as compared to respective 10× control medium treated cells upon tbOH damage (FIG. 6a) whereas at doses of 10% & 50% resulted in 39% & 36% restoration of elastin levels respectively as compared to their respective 10× control medium as seen in FIG. 6b. For hyaluronic acid levels, 10×CM doses of 5% & 10% leads to 17% & 57% of restoration of hyaluronic acid levels respectively as compared to the their respective 10× control medium (FIG. 6c). Similarly, post treatment of 10×CM leads to 59% & 85% increase in Cyclin B 1 levels at doses of 5% & 50% respectively as demonstrated in FIG. 6d, when compared to their respective 10× control medium.

### Conclusion:

Bone marrow mesenchymal stem cell conditioned media possess the potential to regain structural framework to treat damaged skin, to restore elasticity and retain moisture in damaged/aged skin, prevent loss of skin structure and reverse senescence by increasing cyclin B1 levels caused by oxidative stress.

### Example 7

### Protective Effects of Conditioned Medium Generated by Process-1 Against Oxidative Damage to Collagen

The protective effects of 10×CM generated by process-2 against collagen degradation caused by oxidative damage in dermal fibroblast cells upon treatment with 600 μM of H₂O₂ for 2 h is evaluated by measuring levels of collagen 48 h after H₂O₂ damage. HFF-1 cells are treated with different doses of 10×CM and 10× control medium for 24 h prior to H₂O₂ damage.

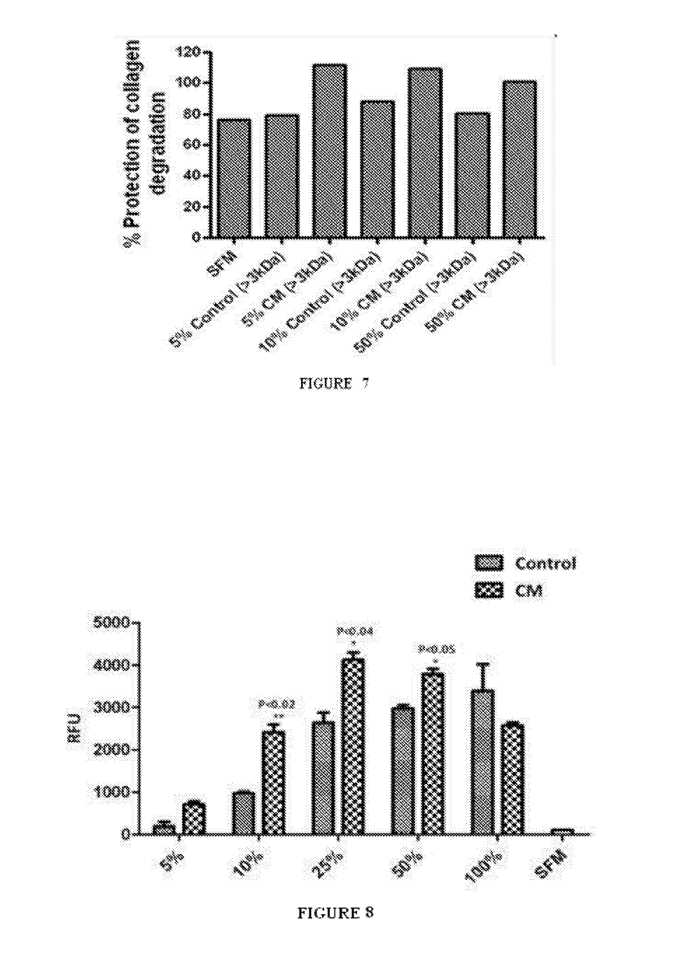

Treatment of HFF-1 cells with 10×CM leads to 33%, 21% and 20% protection against collagen degradation at the doses of 5%, 10% and 50% respectively as compared to respective 10× control medium treated cells upon H₂O₂ damage (FIG. 7). Hence, 10×CM possess the ability to protect collagen degradation induced by oxidative stress and has the potential to prevent loss of skin structure caused by oxidative stress.

### Example 8

### HFF Proliferation Assay

About 2000 HFF cells are seeded per well in a 96-well micro-titre plate in 1×DMEM+15% FBS. On the next day, the cells are serum starved in 1×DMEM+0.1% FBS for 24 hours. Post serum starvation, the cells are treated with different doses of 1×/10× of control medium/CM generated by process 2. MTT assay is performed 6 days post-addition of the control/conditioned medium. The assay employs a dye that is used for measuring the viability and proliferation of the cells colorimetrically.

This assay indicates the proliferation potential of Human fibroblast cells in the presence of conditioned media (FIG. 8). The proliferation potential of HFF 15 found to be the highest when 25% of 1×CM is used. It represents the ability of skin cells to grow new cells contributing to youthful skin.

### Example 9

### Migration Assay or the In-Vitro Scratch Assay

The in vitro scratch assay is an easy, low-cost and well-developed method to measure cell migration in vitro. The assay is particularly suitable for studies on the effects of cell-matrix and cell—cell interactions on cell migration, it mimics cell migration and enables imaging of live cells during migration to monitor intracellular events if desired. The basic steps involve creating a “scratch” in a cell monolayer, capturing the images at the beginning and at regular intervals during cell migration to close the scratch, and comparing the images to quantify the migration rate of the cells.

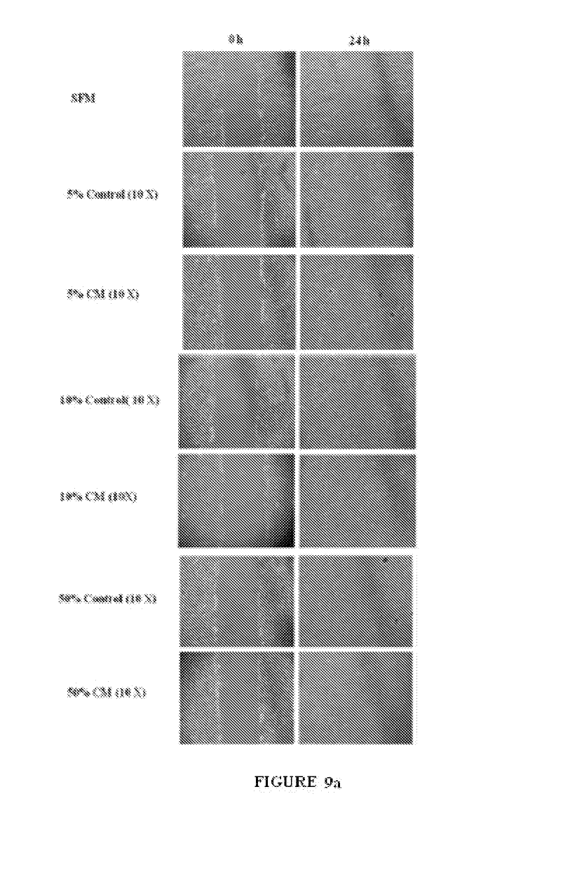

About 250000 HFF cells are seeded per well in a 6-well plate in 1×DMEM+15% FBS in order to achieve more than 90% confluency. On the next day cells are serum starved in 1×DMEM+0.1% FBS for 24 h. Post serum starvation, scratches (straight line) are created in the middle of cell monolayer using tip of sterile 200 μL pipette tip. Cells are washed twice using 1×DMEM followed by treatment with 5%, 10% and 50% of 10× of control medium and CM generated by process 2 for 48 h. Images of the scratches are taken at 0 h and 24 h at three points in single well and distance of scratch closure is measured using ImageJ software (FIGS. 9 a and 9 b).

The results indicate 65% migration of fibroblast cells in the presence of 5%, 10% and 50% conditioned media compared to 30-40% with control media (FIG. 9b). Hence, this indicates the ability of self-renewal of skin cells on treatment with conditioned media.

### Example 10

### Cytoprotective Viability and Restorative Effects of CM

Cytoprotective viability restorative effects of 10×CM generated by process 2 against oxidative damage in dermal fibroblast cells upon treatment with 300 μM of tert-butyl hydroperoxide (tbOH) for 2 h and post treatment of damaged fibroblast with 10×CM for 48 h is evaluated by MTT assay. Treatment of HFF-1 cells with 300 μM of tbOH leads to 35% decrease in cellular viability in SFM treated as compared to untreated cells.

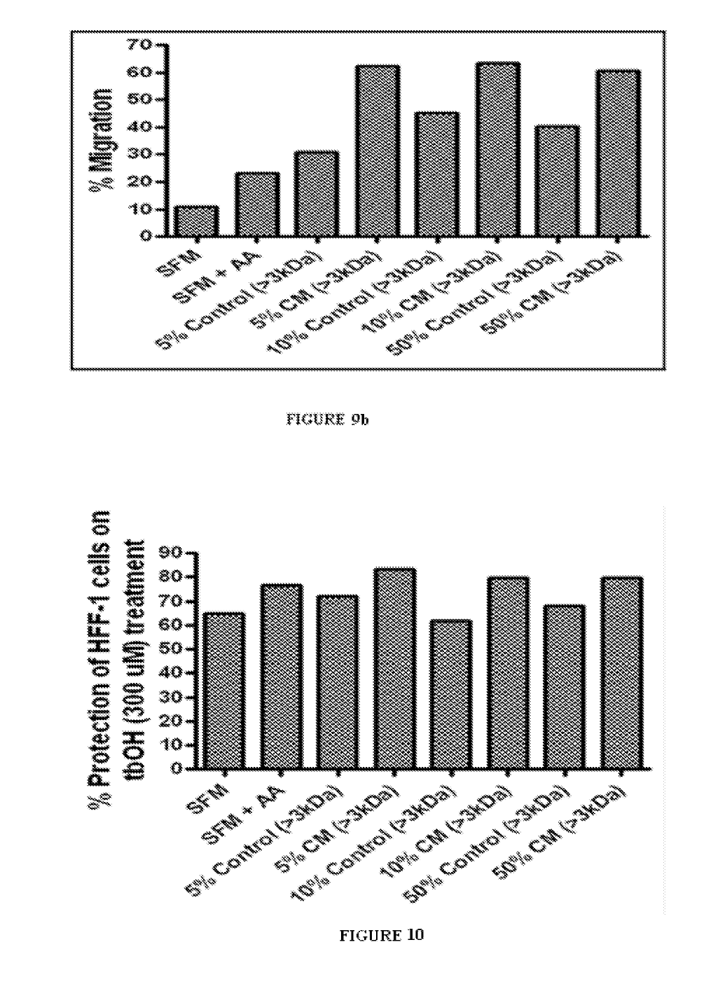

The cytoprotection obtained with 10×CM is 11%, 18% and 12% higher at the doses of 5%, 10% and 50% respectively as compared to respective 10× control medium treated cells against tbOH damage (FIG. 10). In this assay, serum free medium with ascorbic acid (SFM+AA) is used as a positive control. Treatment of HFF-1 cells with 100 μM ascorbic acid (AA) also leads to 12% cytoprotection against tbOH damage. Treatment of damaged HFF-1 cells with 10×CM demonstrates cytoprotective/cellular viability restorative abilities at all the doses tested. 10×CM has shown a trend in the ability to restore the viability of oxidative stressed fibroblast cells at the concentrations 5%, 10% and 50%. Maximum activity is observed at 10% 10×CM. Hence, 10×CM has the ability to restore the viability of oxidative stressed fibroblasts cells and hence possesses the potential to restore skin health.

### Example 11

### In-Vitro Proof of Principle on the Anti-Wrinkling Effect of CM

Even small amounts of UV radiation trigger the processes that can cause wrinkles. Sunlight damages elastin, a protein in the skin that normally maintains the springiness and strength of tissue beneath the skin. In response to this sun-induced elastin damage, large amounts of metalloproteinase are generated which may result in collagen damage. This results in the formation of disorganized collagen fibers known as solar scars. When the skin repeats this imperfect rebuilding process over and over wrinkles develop. In addition, UVB exposure induces cell death by activating apoptotic cell death pathways such as increase in caspase 3 whose activation indicates mitochondrial mediated apoptotic pathway activation. DNA damage also results from UVB exposure, with prominent UV-induced lesions on DNA resulting in CPD (cyclobutane pyrimidine dimer) formation. Further, the activation of caspase 3 and increase in cellular DNA damage results in malignant changes in the epidermis and disorganization in the dermal matrix leading to the appearance of wrinkled skin.

In this study, the restorative effect on elastin synthesis, anti-apoptotic effects against caspase 3 activation and DNA lesion repair effect of 10×CM against UVB exposure in dermal fibroblast cells is analyzed. Post serum starvation, HFF-1 cells are treated with 100 mJ/cm² of UVB irradiation for 2 hours. This was followed by exposure of different doses of 10×CM obtained from culturing of MSCs derived from three donors, when pooled together and cultured by following Process 1 and control medium for 48 hours. Elastin levels are estimated in culture supernatant by ELISA method. For estimation of caspase 3 activation, cells are lysed in cell lysis buffer to make cell lysates to estimate protein levels using BCA (biocinchoninic acid assay) protein estimation. The caspase 3 activity is measured using ELISA. Similarly, the level of CPD formation was estimated by ELISA.

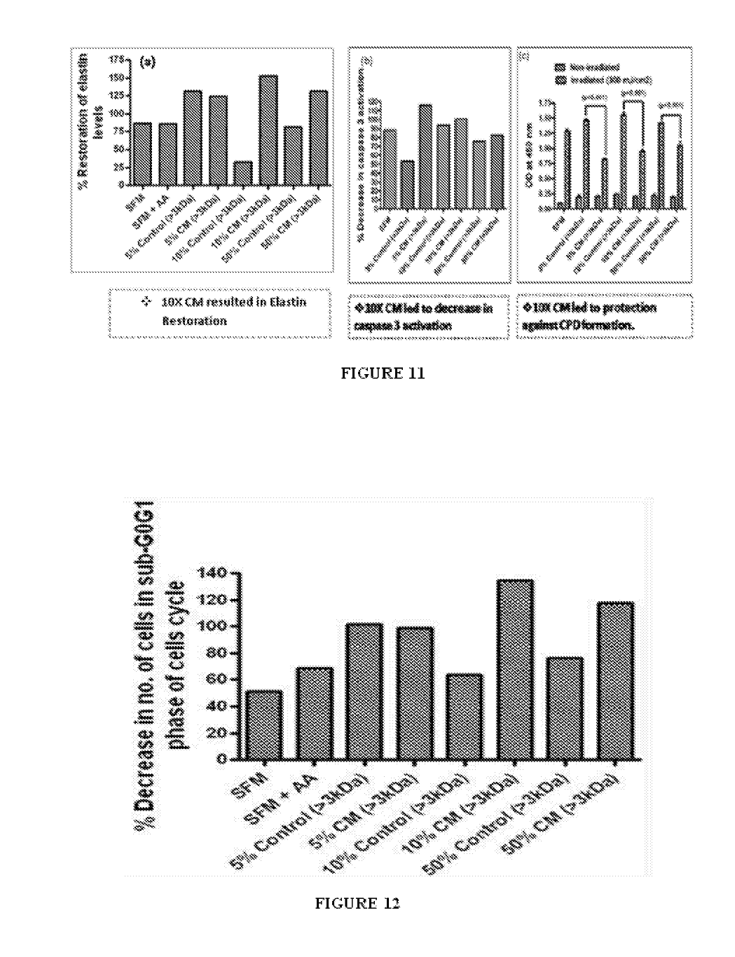

Post treatment of UVB irradiated HFF-1 cells with 10×CM at doses of 10% & 50%, 10×CM leads to 20% & 50% restoration of elastin levels respectively as compared to their respective control medium (FIG. 11a). Treatment with 10×CM at doses of 5% leads to 63% decrease in caspase 3 activation as seen in FIG. 11b, whereas considerable extent of protection against DNA lesions (CPD formation) is seen at all the doses of CM tested (FIG. 11c) when compared to their respective control medium. The highest level of protection against CPD damage is observed at a dose of 5% 10×CM.

### Conclusion:

Bone marrow mesenchymal stem cell derived conditioned media aids in skin structure building/strengthening effects, skin tightening, retention of elasticity, protects against UVB induced apoptosis and possess DNA repairing properties against UVB induced DNA lesions.

### Example 12

### Anti-Apoptotic Effects of CM Against Oxidative Stress Induced Apoptosis in Dermal Fibroblast Cells

The percentage of cells in sub-G0/G1 phase of cell cycle indicates apoptotic population of cells. The anti-apoptotic effect of 10×CM obtained by Process-1 against UVB induced apoptosis in dermal fibroblast cells upon treatment with 230 mJ/cm2 UVB is evaluated by analyzing the percentage of cells in sub-G0/G1 phase of cell cycle 48 h after irradiation. The treatment of human dermal fibroblast cells with 300 mJ/cm2 UVB leads to 55% increase in number of cells in sub-G0/G1 phase of cells cycle.

The treatment of HFF-1 cells with 5% 10×CM does not impart any decrease in number of cells in sub-G0/G1 phase of cell cycle as compared to respective control medium treated cells. Whereas, the treatment of 10×CM leads to 70% and 30% decrease in number of cells in sub-G0/G1 phase of cell cycle at the doses of 10% and 50% respectively as compared to respective 10× control medium treated cells upon UVB irradiation (FIG. 12). Hence, CM protects human dermal fibroblast cells against UVB induced apoptosis at higher doses (10% and 50%).

The conditioned medium produced from the above process is formulated for topical administration. The CM is formulated into but not limited to oily suspensions, hydrogel, nanogel, liposome, wet tissues, ointment, patch, gel, lotion, serum, emulsion, creams, spray, drops, and other known formulation in art can be used or any combination thereof.

### Example 13

### Formulations of the Conditioned Media Rich in Bioactive Factors

### 1. Gel Formulation:

### Procedure:

1. Deionised water is taken in a beaker and EDTA is added to the water with continuous stirring.
2. Methyl paraben and propyl paraben are added and stirred for 5 min.
3. Accurately weighed Conditioned media (Drug) is added into the solution and stirred until it is dispersed uniformly. Gel forming polymer is added slowly with continuous stirring to the above solution until it forms a viscous gel.

### Examples of Gel Formulations:

TABLE 5

| Sl. | | Weight | Weight | | |
|---|---|---|---|---|---|
| No. | Ingredients | (%) | (g or mg) | | Use |
| 01 | Conditioned media/Active | 9.8 | 9.8 | g | Active |
| | media | | | | ingrentient |
| 02 | Hydroxy ethyl cellulose | 2 | 2 | g | Thickening and |
| | | | | | stablizing agent |
| 03 | EDTA | 0.05 | 50 | mg | Chelating agent |
| 04 | Methyl paraben | 0.2 | 200 | mg | Antimicrobial |
| | | | | | agent |
| 05 | Propyl paraben | 0.02 | 20 | mg | Antibacterial |
| | | | | | agent |
| 06 | Deionised water | qs to | 100 | g | solvent |
| | | 100% | | | |

TABLE 6

| Sl. | | Weight | Weight | | |
|---|---|---|---|---|---|
| No. | Ingredients | (%) | (g or mg) | | Use |
| 01 | Conditioned media | 9.8 | 9.8 | g | Active |
| | | | | | ingrentient |
| 02 | Hydroxy propyl cellulose | 2 | 2 | g | Thickening and |
| | | | | | stablizing agent |
| 03 | EDTA | 0.05 | 50 | mg | Chelating agent |
| 04 | Methyl paraben | 0.2 | 200 | mg | Antimicrobial |
| | | | | | agent |
| 05 | Propyl paraben | 0.02 | 20 | mg | Antibacterial |
| | | | | | agent |
| 06 | Deionised water | qs to | 100 | g | solvent |
| | | 100% | | | |

TABLE 7

| | | Weight | Weight | | |
|---|---|---|---|---|---|
| Sl. No. | Ingredients | (%) | (g or mg) | | Use |
| 01 | Conditioned media | 9.8 | 9.8 | g | Active |
| | | | | | ingrentient |
| 02 | Hydroxy propyl | 2.7 | 2.7 | g | Thickening and |
| | methyl cellulose | | | | stablizing agent |
| 03 | EDTA | 0.05 | 50 | mg | Chelating agent |
| 04 | Methyl paraben | 0.2 | 200 | mg | Antimicrobial |
| | | | | | agent |
| 05 | Propyl paraben | 0.02 | 20 | mg | Antibacterial |
| | | | | | agent |
| 06 | Deionised water | qs to 100% | 100 | g | solvent |

TABLE 8

| | | Weight | Weight | | |
|---|---|---|---|---|---|
| Sl. No. | Ingredients | (%) | (g or mg) | | Use |
| 01 | Conditioned media | 9.8 | 9.8 | g | Active |
| | | | | | ingrentient |
| 02 | Carbopol | 6 | 6 | g | Thickening and |
| | | | | | stablizing agent |
| 03 | EDTA | 0.05 | 50 | mg | Chelating agent |
| 04 | Methyl paraben | 0.2 | 200 | mg | Antimicrobial |
| | | | | | agent |
| 05 | Propyl paraben | 0.02 | 20 | mg | Antibacterial |
| | | | | | agent |
| 06 | Deionised water | qs to 100% | 100 | g | solvent |

### Physicochemical and In-Vitro Evaluation of Formulations:

The anti-wrinkle formulations are evaluated for physicochemical properties and in-vitro screening studies. Based on the feasibility, the initial in-vitro screening studies are conducted in Franz diffusion cell using rat skin or dialysis membrane. For the final optimized formula cadaver skin is used. During the in-vitro screening studies receptor sample is collected at specified time intervals (0, 0.10, 0.20, 0.30, 0.45, 1, 2, 4, 6, 8, 10, 12 and 24 hours) and it is analyzed by UV spectroscopy at 280 and 200 nm.

The proteins (growth factors) present in the receptor sample, 1 absorb ultraviolet light with absorbance maxima at 280 and 200 nm. These parameters is compared with marketed and placebo formulations.

| | |
|---|---|
| Physicochemical evaluation | Appearance |
| | Color |
| | Odour |
| | pH |
| | Viscosity |
| | Spreadability |
| In-vitro screening using Franz | Total protein content at specified time |
| diffusion cell | interval at 280 and 200 nm |

### Stability Studies:

The final formulation is packed and labeled in a suitable container meant for marketing and kept for stability under various conditions as per suitable ICH guidelines. At certain decided interval referred as stability time point samples are withdrawn and all the physicochemical evaluation (PCE) is performed.

All the formulations and the corresponding data provided within the purview of this invention by way of specific examples have a standard deviation of ±10%.

All the above gels are evaluated for the following physical parameters:
1. pH
2. Spreadability
3. Appearance

Based on the physical evaluation, a gel formulation with appropriate viscosity and spreadability is selected for animal studies. Further the gels are placed in stability chambers and evaluated for the above physical parameters at specified time points as per the proposal.

TABLE A
Initial sample results

| Table Number | pH | Spreadability | Appearance |
|---|---|---|---|
| Table 5 | 5.21 | 13.86 | Pale yellow coloured viscous gel |
| Table 6 | 4.05 | 13.86 | Pale yellow coloured viscous gel |
| Table 7 | 5.45 | 16.0 | Pale yellow coloured viscous gel |
| Table 8 | 2.45 | 11.46 | Pale yellow coloured viscous gel |

TABLE B
Evaluation of gels placed on stability at
40° C./75% RH at 2 weeks' time point

| Table number | pH | Spreadability | Appearance |
|---|---|---|---|
| Table 5 | 5.13 | 13.33 | Pale yellow coloured viscous gel |
| Table 6 | 4.56 | — | Half melted into liquid half |
| | | | remains as white solid. |
| Table 7 | 5.49 | 16.00 | Pale yellow coloured viscous gel |
| Table 8 | 2.50 | 10.13 | Pale yellow coloured viscous gel |

### Evaluation of Gels Placed on Stability at 2°-8° C., 25° C./60% RH and 40° C./75% RH at 1 Month Time Point

TABLE C
pH studies:

| | pH | | |
|---|---|---|---|
| Batch number | 2°-8° C. | 25° C./60% RH | 40° C./75% RH |
| Table 5 | 4.58 | 5.12 | 5.05 |
| Table 6 | 3.70 | 4.62 | 4.92 |
| Table 7 | 5.55 | 5.71 | 5.64 |
| Table 8 | 2.55 | 2.61 | 2.93 |

TABLE D
Spreadability test:

| | Spreadability | | |
|---|---|---|---|
| Batch number | 2°-8° C. | 25° C./60% RH | 40° C./75% RH |
| Table 5 | 13.33 | 13.33 | 13.33 |
| Table 6 | 13.50 | 13.60 | — |
| Table 7 | 16.00 | 16.00 | 16.00 |
| Table 8 | 10.13 | 10.13 | 10.13 |

TABLE E
Physical appearance:

| Batch | Appearance | | |
|---|---|---|---|
| number | 2°-8° C. | 25° C./60% RH | 40° C./75% RH |
| Table 5 | Pale yellow | Pale yellow | Pale yellow coloured |
| | coloured viscous | coloured viscous | viscous gel |
| | gel | gel | |
| Table 6 | Pale yellow | Pale yellow | Half melted into liquid |
| | coloured viscous | coloured viscous | half remains as white |
| | gel | gel | solid. |
| Table 7 | Pale yellow | Pale yellow | Pale yellow coloured |
| | coloured viscous | coloured viscous | viscous gel |
| | gel | gel | |
| Table 8 | Pale yellow | Pale yellow | Pale yellow coloured |
| | coloured viscous | coloured viscous | viscous gel |
| | gel | gel | |

### Vehicle Alone Formulations or the Base Formulation

TABLE 9

| | | | Weight | |
|---|---|---|---|---|
| Sl. No. | Ingredients | Weight (%) | (g or mg) | |
| 01 | Conditioned media | 00 | 00 | g |
| 02 | Hydroxy ethyl cellulose | 2 | 4 | g |
| 03 | EDTA | 0.05 | 100 | mg |
| 04 | Methyl paraben | 0.2 | 400 | mg |
| 05 | Propyl paraben | 0.02 | 40 | mg |
| 06 | Deionised water | qs to 100% | | |

### Formulations Using Conditioned Media—Drug (30%)

TABLE 10

| | | | Weight | |
|---|---|---|---|---|
| Sl. No. | Ingredients | Weight (%) | (g or mg) | |
| 01 | Conditioned media | 30 | 60 | g |
| 02 | Hydroxy ethyl cellulose | 2 | 4 | g |
| 03 | EDTA | 0.05 | 100 | mg |
| 04 | Methyl paraben | 0.2 | 400 | mg |
| 05 | Propyl paraben | 0.02 | 40 | mg |
| 06 | Deionised water | qs to 100% | | |

### Formulations Using Conditioned Media—Drug (70%)

TABLE 11

| | | | Weight | |
|---|---|---|---|---|
| Sl. No. | Ingredients | Weight (%) | (g or mg) | |
| 01 | Conditioned media | 70 | 140 | g |
| 02 | Hydroxy ethyl cellulose | 2 | 4 | g |
| 03 | EDTA | 0.05 | 100 | mg |
| 04 | Methyl paraben | 0.2 | 400 | mg |
| 05 | Propyl paraben | 0.02 | 40 | mg |
| 06 | Deionised water | qs to 100% | 200 | g |

### Evaluation of Vehicle Alone Formulations or the Base Formulation of Tables 9-11.

All the above gels are evaluated for the following physical parameters:
1. pH
2. Spreadability
3. Appearance

Based on the physical evaluation, a gel formulation with appropriate viscosity and spreadability is selected for animal studies. Further the gels are placed in stability chambers and evaluated for the above physical parameters at specified time points as per the proposal.

TABLE A
Initial sample results

| Table Number | pH | Spreadability | Appearance |
|---|---|---|---|
| Table 9 | 3.57 | 13.60 | Colourless viscous gel |
| Table 10 | 5.35 | 13.33 | Pale yellow coloured viscous gel |
| Table 11 | 6.92 | 13.33 | Reddish coloured gel |

TABLE B
Evaluation of gels placed on stability at
40° C./75% RH at 2 weeks' time point

| Table number | pH | Spreadability | Appearance |
|---|---|---|---|
| Table 9 | 3.61 | 13.60 | Colourless viscous gel |
| Table 10 | 5.30 | 13.33 | Pale yellow coloured viscous gel |
| Table 11 | 6.81 | 13.60 | Reddish coloured gel |

### Evaluation of Gels Placed on Stability at 2°-8° C., 25° C./60% RH and 40° C./75% RH at 1 Month Time Point

TABLE C
pH studies:

| | pH | | |
|---|---|---|---|
| Batch number | 2°-8° C. | 25° C./60% RH | 40° C./75% RH |
| Table 9 | 3.60 | 3.58 | 3.59 |
| Table 10 | 5.35 | 5.32 | 5.31 |
| Table 11 | 6.85 | 6.86 | 6.83 |

TABLE D
Spreadability test:

| | Spreadability | | |
|---|---|---|---|
| Batch number | 2°-8° C. | 25° C./60% RH | 40° C./75% RH |
| Table 9 | 13.60 | 13.60 | 13.60 |
| Table 10 | 13.33 | 13.33 | 13.33 |
| Table 11 | 13.33 | 13.60 | 13.60 |

TABLE E
Physical appearance:

| | Appearance | | |
|---|---|---|---|
| | | | 40° C./ |
| Batch number | 2°-8° C. | 25° C./60% RH | 75% RH |
| Table 9 | Colourless viscous | Colourless viscous | Colourless |
| | gel | gel | viscous gel |
| Table 10 | Pale yellow | Pale yellow | Pale yellow |
| | coloured viscous | coloured viscous | coloured |
| | gel | gel | viscous gel |
| Table 11 | Reddish coloured | Reddish coloured | Reddish |
| | gel | gel | coloured gel |

### Evaluation of Gels Placed on Stability at 2°-8° C., 25° C./60% RH and 40° C./75% RH at 3 Month Time Point

TABLE F
pH studies:

| | pH | | |
|---|---|---|---|
| Batch number | 2°-8° C. | 25° C./60% RH | 40° C./75% RH |
| Table 9 | 3.02 | 2.99 | 3.59 |
| Table 10 | 5.60 | 5.52 | 5.31 |
| Table 11 | 6.87 | 6.72 | 6.83 |

TABLE G
Spreadability test:

Spreadability

| Batch number | 2°-8° C. | 25° C./60% RH |
|---|---|---|
| Table 9 | 13.33 | 14.13 |
| Table 10 | 13.60 | 14.40 |
| Table 11 | 13.60 | 14.66 |

TABLE H
Physical appearance:

Appearance

| Batch number | 2°-8° C. | 25° C./60% RH |
|---|---|---|
| Table 9 | Colourless viscous | Colourless viscous |
| | gel | gel |
| Table 10 | Pale yellow | Pale yellow |
| | coloured viscous | coloured viscous |
| | gel | gel |
| Table 11 | Reddish coloured | Reddish coloured |
| | gel | gel |

### 2. Serum Formulation:

### Conditioned Medium for Skin Health Including Anti-Ageing and Skin Rejuvenation

Concentrated conditioned medium is formulated into a serum for cosmetic application. The cosmetic application includes use the serum formulation for general skin health including, anti-ageing, anti-wrinkling, anti-scaring and for skin revival and skin rejuvenations.

The BM-MSC conditioned media (CM) based anti-wrinkle serum is prepared by suitable preparation method with compatible ingredients to stabilize and to protect varies bioactive factors present in the conditioned media. Each batch formula is prepared with 1×, 3× and 7× concentration of the BMMSC conditioned media. The table below illustrates the 4 different type of serum formulation.

### Stability:

Since active ingredient is mainly proteins it is mandatory to preserve the function of the proteins which is present in the final product. Hydroxyethylcellulose, amino acids selected from a group comprising L-Arginine and L-Glutamic acid or a combination thereof at a concentration ranging from about 50 mM to about 100 mM acts as stabilizer to preserve the active ingredient.

TABLE 12
Serum formulation A

| | | | Grade/ |
|---|---|---|---|
| Ingredients | Use | Quantity (%) | Supplier |
| Conditioned media | Growth factor | 10, 30 or 70 | Stempeutics |
| | media - main | | |
| | active | | |
| Hydroxyethylcellulose | Thickener, stabilizer | 3.0 | Sigma Fluka |
| Glycerin | Moisturizer | 3.0 | USP/Sigma |
| DL-Panthenol | Emollient | 2.0 | Sigma |
| | | | Aldrich |
| Phenoxyethanol | Preservative | 0.5 | Himedia |
| Allantoin | Soothing agent | 1.0 | Sigma |
| PEG | Permeation | 3.0 | Sigma/ |
| | enhancer | | Kollisolv |
| Purified water | Solvent | Q.S | MCOPS |

TABLE 13
Serum formulation B

| | | | Grade/ |
|---|---|---|---|
| Ingredients | Use | Quantity (%) | Supplier |
| Conditioned media | Growth factor | 10, 30 or 70 | Stempeutics |
| | media - main | | |
| | active | | |
| Hydroxyethylcellulose | Thickener, stabilizer | 3.0 | Sigma Fluka |
| Glycerin | Moisturizer | 3.0 | USP/Sigma |
| DL-Panthenol | Emollient | 2.0 | Sigma |
| | | | Aldrich |
| Allantoin | Soothing agent | 1.0 | Sigma |
| PEG | Permeation | 3.0 | Sigma/ |
| | enhancer | | Kollisolv |
| Xanthan gum | Stabilizer | 1.0 | Himedia |
| Sodium PCA | Humectant | 1.5 | Himedia |
| Gluconolactone | Moisturizer/ | 1.0 | Sigma |
| | antioxidant | | |
| Sodium Benzoate | Preservatives | 0.5 | Sigma |
| Sodium Hydroxide | pH balancer | Q.S | Himedia |
| Purified water | Solvent | Q.S | MCOPS |

TABLE 14
Serum formulation C

| Serial | | | |
|---|---|---|---|
| No. | Name of the Ingredient | Function | % w/w |
| 1. | Conditioned media | Active ingredient | 10-30 |
| 2. | Purified water | Vehicle | q.s. to 92.1% |
| 3. | Glycerin | Tightening & moisturizing | 1.0 |
| 4. | Phenoxyethanol and Ethylhexylglycerin | Preservative | 1.0 |
| | (Euxyl PE9010) | | |
| 5. | Xanthan Gum | Viscosity increasing agent | 0.5 |
| 6. | Sodium Polyacrylate (and) Caprylic/ | Viscosity increasing agent | 0.2 |
| | Capric Triglyceride (and) Mineral oil | | |
| | (and) Tri (PPG-3 Myristyl ether) Citrate | | |
| | (and) SorbitanLaurate (and) Trideceth-6 | | |
| | (OptaSense RMA-IS) | | |
| 7. | PEG-75 Lanolin (Solan E) | Emollient | 0.2 |

TABLE 15
Serum formulation D

| Serial | | | |
|---|---|---|---|
| No. | Ingredient | Activity | % w/w |
| 1 | Conditioned media | Active ingredient | 10%-30% |
| 2. | Purified water | Solvent | q.s to |
| | | | 91.3% |
| 3 | Glycerin | Moisturizer | 1 |
| 4 | Phenoxyethanol and | Preservative | 1 |
| | Ethylhexylglycerin | | |
| | (Euxyl PE9010) | | |
| 5 | Xanthan Gum | Stabilizer | 0.5 |
| 6 | Beta Glucan | Bulking Agent; | 0.4 |
| | | Skin- | |
| | | Conditioning | |
| | | Agent | |
| 7 | Sodium Polyacrylate (and) | Emulsifier/ | 0.2 |
| | Caprylic/Capric Triglyceride | thickening | |
| | (and) Mineral Oil (and) Tri (PPG-3 | agent | |
| | Myristyl ether) Citrate (and) | | |
| | Sorbitan Laurate (and) Trideceth-6 | | |
| | (Optasense RMA-IS) | | |
| 8 | PEG-75 Lanolin (Solan E) | Water soluble | 0.2 |
| | | emollient, O/W | |
| | | Emulsifier, | |
| | | Solubiliser and | |
| | | Wetting agent | |
| 9 | Sodium Hyaluronate | Permeation | 0.2 |
| | | Enhancer | |
| 10 | D-Panthenol | Moisturizer | 0.2 |

TABLE 16
Liposomal Serum Formulation-E
The anti-aging liposomal serum formulation comprises of conditioned
medium rich in bioactive factor secreted by mesenchymal stem/stromal
cell which can promote youthful skin.

| Ingredients | Type/Activity | Percentage |
|---|---|---|
| Conditioned Media | Active | 1-5 |
| Phosphatidylcholine | Lipid | 5-95 |
| Cholesterol | Surfactant | 1-50 |
| Essential Oil | Stabilizer | 3-90 |
| DL-α-Tocopherol acetate | Antioxidant | 0.25-25 |
| Hydroxyethyl cellulose | Thickener | 0.5-15 |
| Glycerine | Moisturizer | 0.5-15 |
| Allantoin | Soothing agent | 0.10-3 |
| Phenoxyethanol | Preservative | 0.10-3 |
| DL-Panthenol | Emollient | 0.10-5 |
| PEG | Permeation enhancer | 0.5-15 |
| Cyclodextrin | Stabilizer | 0.5-10 |
| Purified water | Solvent | 10-95 |
| Fragrance | Fragrant | 0.1-1 |

### Physicochemical Evaluation of Formulations:

The anti-wrinkle formulations are evaluated for physicochemical properties and in-vitro screening studies. Based on the feasibility, the initial in-vitro screening studies are conducted in Franz diffusion cell using rat skin or dialysis membrane. For the final optimized formula cadaver skin is used. During the in-vitro screening studies receptor sample is collected at specified time intervals (0, 0.10, 0.20, 0.30, 0.45, 1, 2, 4, 6, 8, 10, 12 and 24 hours) and it is analyzed by UV spectroscopy at 280 and 200 nm.

The proteins (growth factors) present in the receptor sample, 1 absorb ultraviolet light with absorbance maxima at 280 and 200 nm. These parameters is compared with marketed and placebo formulations.

TABLE 17
Physicochemical evaluation of formulations

| Physicochemical evaluation | Appearance |
|---|---|
| | Color |
| | Odour |
| | pH |
| | Viscosity |
| | Spreadability |
| In-vitro screening using Franz | Total protein content at specified time |
| diffusion cell | interval at 280 and 200 nm |

### Stability Studies:

The final formulation is packed and labeled in a suitable container meant for marketing and kept for stability under various conditions as per suitable ICH guidelines. At certain decided interval referred as stability time point samples are withdrawn and all the physicochemical evaluation (PCE) is performed.

TABLE 18

| | Months | | | |
|---|---|---|---|---|
| Storage condition (Temp/RH) | Initial | 1 | 2 | 3 |
| 40° C. ± 2° C./75% RH ± 5% RH | PCE | PCE | PCE | PCE |
| 25° C. ± 2° C./60% RH ± 5% RH | PCE | PCE | PCE | PCE |
| 5° C. ± 3° C. | PCE | PCE | PCE | PCE |

All the formulations and the corresponding data provided within the purview of this invention by way of specific examples have a standard deviation of ±10%.

In the embodiment the formulations analysed for in vivo efficacy are:

TABLE 19
Formulation 1

| Ingredient | Qty (%) |
|---|---|
| Conditioned media from process 2 (Active) | 10 |
| Purified Water (Solvent) | q.s to 82.89% |
| Glycerin Moisturizer | 0.9 |
| Euxyl PE9010 Preservative | 0.9 |
| Xanthan Gum Stabilizer | 0.45 |
| Optasense RMAIS Emulsifier/thickening | 0.18 |
| agent | |
| Solan E Water soluble emollient, O/W | 0.18 |
| Emulsifier, Solubiliser and Wetting agent | |

TABLE 20
Formulation 3

| Ingredient | Qty (%) |
|---|---|
| Conditioned media from process-2 (Active) | 30 |
| Hydroxyethylcellulose Thickener, stabilizer | 1.988 |
| Glycerin Moisturizer | 1.988 |
| DL-Panthenol Emollient | 1.316 |
| Phenoxyethanol Preservative | 0.322 |
| Allantoin Soothing agent | 0.672 |
| PEG Permeation enhancer | 1.988 |
| Purified water Solvent | 61.726 |

TABLE 21
Physiochemical analysis

| | Formulation 1 (F1 | Formulation 3 (FN3- |
|---|---|---|
| Tests | 1X) | 3X) |
| Appearance | Gel | Gel |
| Colour | Pale Yellow | Light Pink |
| Odour | Characteristic | Slight characteristic |
| pH | 5.97 | 7.0-7.5 |
| Viscosity | 10176 | 6038 |
| (cP) | | |
| Spreadability | 29 | 25.53 |
| (gm · cm/sec) | | |

Physico-chemical analyses of selected parameters are assessed. Appearance and colour of the given formulations were tested visually. pH of the given test samples was performed using pH meter. Viscosity is the measure of fluid friction which can be considered as the internal friction resulting when a layer of fluid is made to move in relationship to another layer. It is the measure of the ratio of shearing stress to rate of shear. The viscosity of the given formulation 1 and 3 was tested using a Brookfield Viscometer. The Brookfield Viscometer measures viscosity by measuring the force required to rotate a spindle in a fluid. Spreadability of the given formulation was tested against standard weight applied on the sample glass plate and measuring the area of sample traversed.

### Example 14

### In Vivo Anti-Wrinkling Study

The present study is conducted to evaluate the anti-wrinkling efficacy of the test formulations against pathophysiological changes induced by UVB irradiations in nude mice. In the present study, 6 to 8 weeks old nude mice are selected. All animals except non-irradiated group (G1) are irradiated with UVB up to 7 days. The dose of irradiation is maintained at 150 mJ/cm². Immediately post UVB irradiation, test formulations [as described below] and vehicles (placebo serum formula 1 and placebo serum formula 2) are topically administered to the entire dorsal back of each animal in respective groups. Since UVB exposure leads to pathophysiological changes in animal skin, all the animals are closely observed for appearance of any wrinkles; roughness of skin; loss of water (moisture) and erythema (redness). Hydration of the skin and erythema index (E.I.) are measured using corneometer and mexameter respectively each day before UVB exposure.

A comparison is made between day 1 and 8 for the mean corneometer and mexameter units of test formulation and it is revealed that there is low extent of decrease in skin moisture in groups G5 (21.80%) and G6 (23.86%) as compared to day 1. Formulation 1 applied at dose of 1× concentration (i.e. 10% of the 10×CM) in G5 and formulation 3 applied at dose of 3× (i.e. 30% of the 10×CM) concentration in G6 could substantially prevent UVB induced moisture loss from the skin.

On day 8, extent of increase of E.I. is found to be low in formulation 1 (1× concentration) treated group G5 (2.26%) and formulation 3 (3× concentration) treated group G6 (2.73%) as compared to day 1. Formulation 1 applied at dose of 1× concentration in G5 and formulation 3 applied at dose of 3× concentration in G6 can substantially prevent UVB induced erythema in the skin.

The role of test formulations in reducing the degradative changes in skin upon exposure to UV-B radiation was investigated in nude mice. The degree of protection was assessed using macroscopic and microscopic parameters. Photographic images were recorded to capture the effect of UV-B and the anti-wrinkling potential of the formulations 1 and 3. The camera was clamped on a stand at a fixed distance from the animals and photography was done under aseptic conditions. In this study, the anti-wrinkling efficacy of the formulations was evaluated against pathophysiological changes induced by UV-B irradiations in nude mice. Six to eight weeks old nude mice were selected. All animals except non-irradiated group (control group) were irradiated with UV-B up to 7 days. The dose of irradiation was maintained at 150 mJ/cm². Immediately post UV-B irradiation, formulations 1, and 3 were topically administered to the entire dorsal back of each animal in respective groups. Since UV-B exposure leads to pathophysiological changes in animal skin, all the animals were closely observed for appearance of any wrinkles; laxity and roughness of skin. Untreated (control group) did not indicate wrinkling of skin, however, on UVB treatment, the skin exhibited wrinkling and roughness. On treatment with formulation 1 and 3, the skin condition improved significantly although the animals were UVB treated. There was an exceptional decrease in wrinkling and roughness of skin. Hence, the treatment with the formulation restores skin health and prevents skin damage caused by UVB.

### Allocation of Groups

### Group Treatment

| | |
|---|---|
| G1 | Untreated |
| G2 | UVB treated |
| G3 | Vehicle of Formulation 1 (Placebo) (VF1) |
| G4 | Vehicle of Formulation 3(Placebo) (VF3) |
| G5 | Formulation F1-1X |
| G6 | Formulation FN3-3X |

TABLE 22
Mean Corneometer reading indicative (% change) of skin hydration levels.

| | G1 | G2 | G3 | G4 | G5 | G6 |
|---|---|---|---|---|---|---|
| Day | Untreated | UVB | VF1- | VF-3 | F1-1X | FN3-3X |
| 1 | 100 | 100 | 100 | 100 | 100 | 100 |
| 2 | 99.52 | 88.05 | 98 | 98.64 | 95.44 | 95.13 |
| 3 | 101.29 | 78.47 | 88.82 | 100.35 | 92.3 | 97.02 |
| 4 | 107.83 | 57.62 | 80.85 | 83.79 | 84.81 | 77.82 |
| 5 | 106.52 | 45.51 | 75.91 | 76.35 | 88.87 | 62.9 |
| 6 | 105.56 | 38.68 | 68.1 | 71.13 | 77.28 | 62.15 |
| 7 | 108.38 | 31.81 | 65.4 | 62.95 | 76.28 | 64.03 |
| 8 | 112.41 | 27.72 | 54.66 | 58.9 | 78.2 | 76.54 |

The corneometer reading indicative of skin hydration levels is considered at the end of 8th day which is after 7 days of UVB irradiation according to the experimental design. In the graphical representation, it is observed that there is a gradual decrease in skin hydration levels in all the samples including UVB treated, and placebo samples whereas the skin hydration level is retained to 76-78% at the end of 8th day.

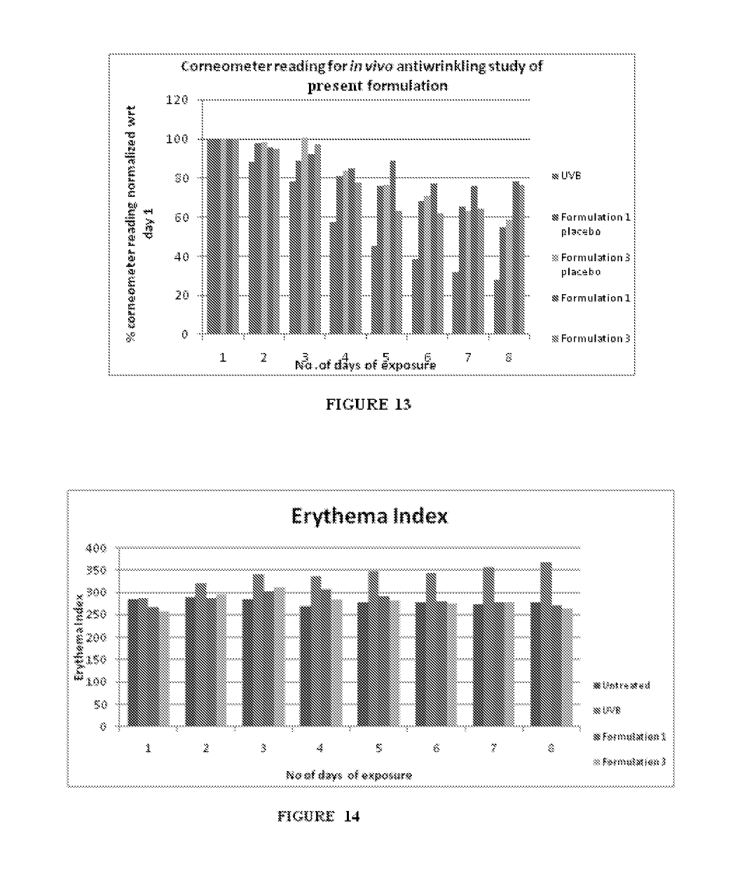

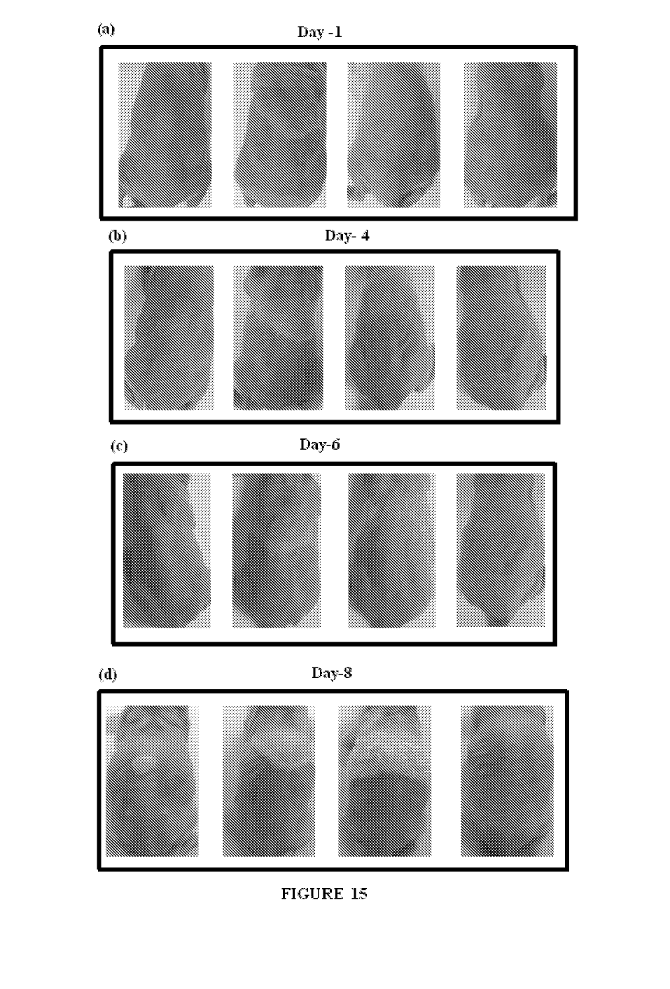

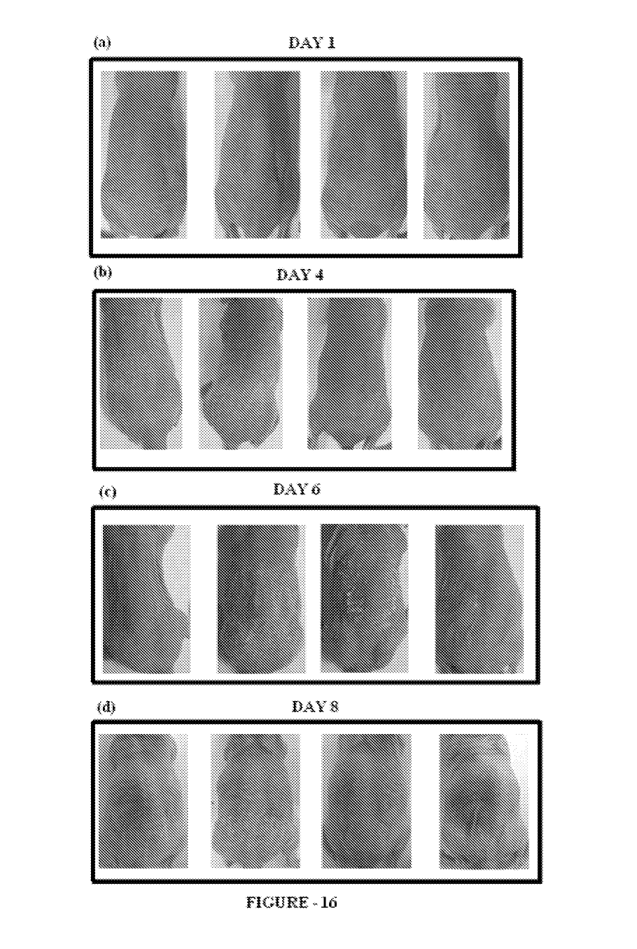

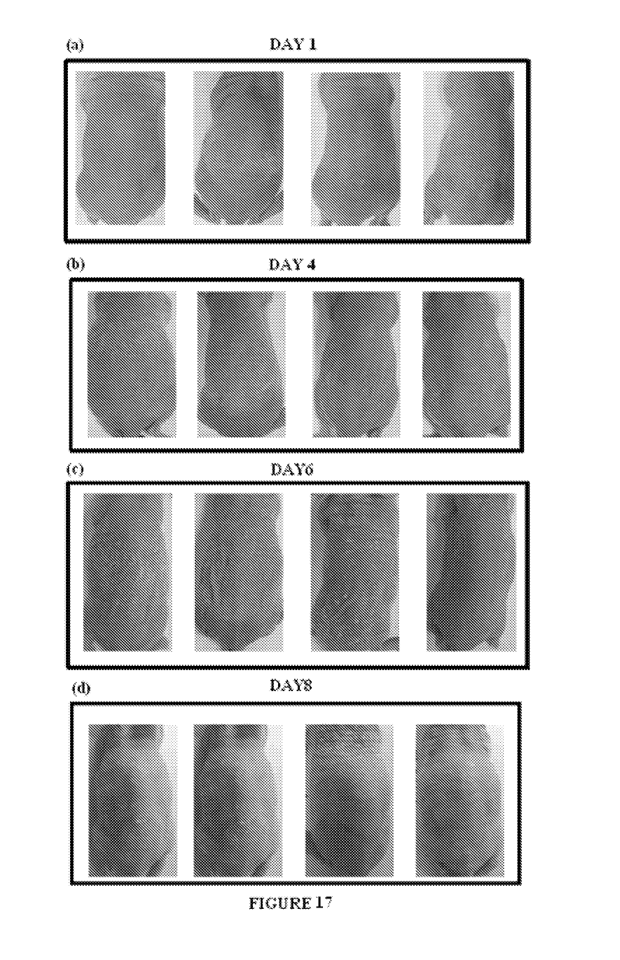

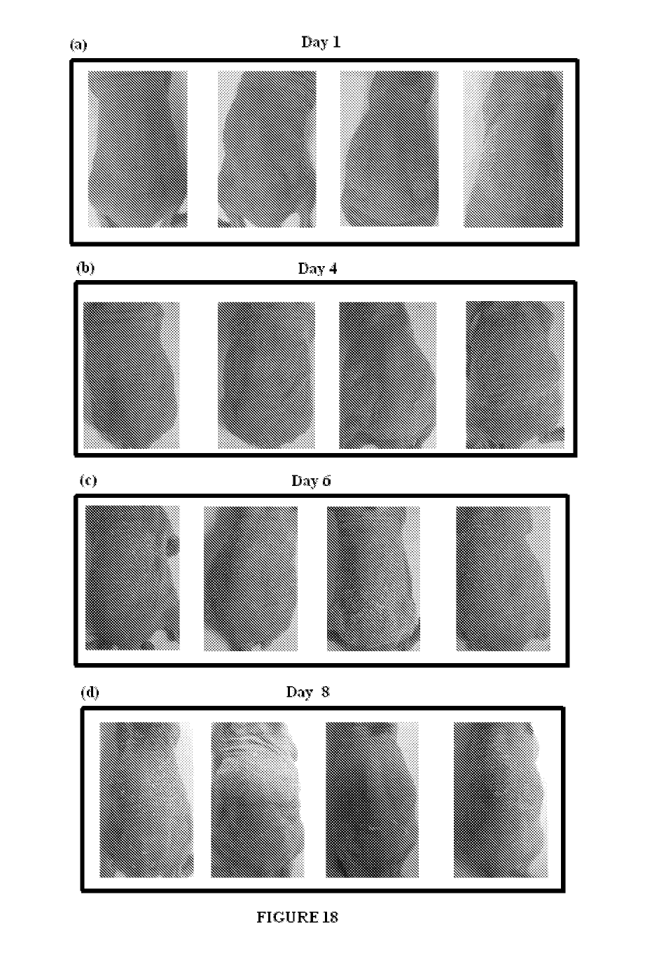

As evident from FIG. 13, at the end of the 8^{th} day, the moisture retention of the formulation treated groups is better than the placebos. From this data, F1-1× has shown better retention of skin hydration levels.

TABLE 23
Mean mexameter readings indicative of erythema index.

| | G1 | G2 | G3 | G4 | G5 | G6 |
|---|---|---|---|---|---|---|
| Day | Untreated | UVB | VF-1 | VF-3 | F1-1X | FN3-3X |
| 1 | 285.4 | 286.8 | 239.2 | 254 | 265.4 | 256.4 |
| 2 | 290 | 321.2 | 254.8 | 262.4 | 287.6 | 296.6 |
| 3 | 285.4 | 340.2 | 264.4 | 275.6 | 302 | 311 |
| 4 | 269.5 | 336.4 | 304.3 | 295 | 306.2 | 284.6 |
| 5 | 278 | 346.2 | 296.5 | 304.6 | 291.2 | 283 |
| 6 | 278.5 | 342 | 295.8 | 301.6 | 280 | 274.6 |
| 7 | 272.3 | 355.2 | 309.8 | 309.4 | 277.8 | 278 |
| 8 | 277 | 366.2 | 300.8 | 297.4 | 271.4 | 263.4 |

The mexameter reading indicative of erythema index is considered at the end of 8th day which is after 7 days of UVB irradiation according to the experimental design. In the graphical representation, it is observed that there is a gradual increase in erythema index in all the samples including UVB treated, and placebo samples whereas the erythema index obtained upon treatment with formulations remains constant to the order of 270 similar to the untreated control.

From this data, it is apparent that both the formulations show lower erythema indices.

### Advantages and Applications

The invention addresses the variation in the levels of bioactive factors produced/secreted by the mesenchymal stem cells from individual donors as compared to that of pooled donors. Certain bioactive factors that are not produced or produced at a very low amount by individual donors are secreted at higher levels in conditioned media obtained from by pooling of mesenchymal stem cells from multiple donors. Hence cytokines and growth factors secreted by pooled donors minimize the heterogeneity seen in conditioned media obtained from individual donors.
The pooling reduced the individual variability of the BMMSC samples. Upon comparing the quantities of bioactive factors produced from three individual donors to that of the three-donor pool sample, it is observed that the variations arising from individual donors are normalized in donor pool. For example, by referring to the data given in the table 2, it is evident that TGF-beta is produced by only one donor (donor 1), and not produced by donors 2 and 3. Therefore TGF-beta is absent in the conditioned media from donors 2 or 3. However pooled condition medium contains TFG-beta. Hence, pooling minimizes or eliminates individual variation.
Pooling also maximizing the probability of generating mesenchymal stromal/stem cells derived conditioned medium representing large number of bioactive factors and others molecules with specific biological function/property. For example conditioned medium having functional VEGF, at a concentration to carry out the required angiogenesis. Also, pooling is done to compensate biological factors not secreted by one of the selected individual being pooled. The present method of obtaining conditioned medium is found to show higher levels of cytokines and growth factor expression necessary for biological/medicinal applications.
The CM based formulation can be used for various cosmetic and therapeutic purposes depending on the expression and secretion of cytokines/GF by screening.
The expression and secretion of specific type of cytokines and GF are used for specific indications. For example, FGF and HGF are important in anti-scarring properties.
The conditioned medium of the present disclosure can also be employed in the form of wet wipes, cosmetic patches and hydrogels.

1. A method of preparing a conditioned medium comprising bioactive factors secreted by mesenchymal stromal cells, said method comprising acts of:
a. culturing the mesenchymal cells in a cell culture medium followed by expanding and harvesting of the cells; and
b. subjecting the harvested cells to a process of:
a. fed batch activation;
b. fed batch activation followed by complete medium change; or
c. complete medium change; or any combination thereof,
to obtain said conditioned medium.

2. The method as claimed in claim 1, wherein the mesenchymal cells are mesenchymal stromal cells or mesenchymal stem cells or a combination thereof; wherein the mesenchymal cells are bone marrow derived mesenchymal cells; and wherein the mesenchymal cells are isolated from individual donors and pooled to obtain pooled mesenchymal cells.

3. (canceled)

4. The method as claimed in claim 1, wherein the mesenchymal cells are seeded and expanded as passage 4 cultures at a seeding density of about 1000 cells/cm² to about 10,000 cells/cm², preferably about 1000 cells/cm²; and wherein the cells are expanded to a confluency of about 50% and subjected to medium change and further cultured till about 80% to about 90% confluency.

5. (canceled)

6. The method as claimed in claim 4, wherein the cells are further seeded and expanded as passage 5 cultures at a seeding density of about 1000 cells/cm² to about 10,000 cells/cm², preferably about 1000 cells/cm² till about 45% to about 50% confluency.

7. The method as claimed in claim 1, wherein the fed batch activation comprise acts of:
a. adding about 500 ml of cell culture medium to the passage 5 cells expanded till about 45% to about 50% confluency; and
b. allowing culturing of the cells till about 80% to about 90% confluency and harvesting the conditioned medium.

8. The method as claimed in claim 1, wherein the fed batch activation followed by—complete medium change comprise acts of:
a. adding about 500 ml of cell culture medium to the passage 5 cells expanded till about 45% to about 50% confluency;
b. allowing culturing of the cells till about 65% to about 70% confluency and subjecting the cells to complete medium change by adding 2 L of cell culture medium; and
c. allowing culturing of the cells till about 80% to about 90% confluency and harvesting the conditioned medium.

9. The method as claimed in claim 1, wherein the complete change of cell culture medium comprise acts of:
a. subjecting the passage 5 cells expanded till about 45% to about 50% confluency to a complete medium change by adding 1.5 L of cell culture medium; and
b. allowing culturing of the cells till about 80% to about 90% confluency and harvesting the conditioned medium.

10. The method as claimed in claim 1, wherein the conditioned medium is concentrated and enriched by ultrafiltration or tangential flow filtration using molecular weight cut-offs of about 1 kDa and above or about 3 kDa and above; and wherein after the concentration, amino acid selected from a group comprising L-Arginine and L-Glutamic acid or a combination thereof is optionally added to the conditioned medium at a concentration ranging from about 50 mM to about 100 mM.

11. The method as claimed in claim 1, wherein said method optionally comprises pooling of the conditioned medium obtained from each of the three processes.

12. The method as claimed in claim 1, wherein the cell culture medium comprises components selected from a group comprising Dulbecco's Modified Eagle Medium-Knock Out, Dulbecco's Modified Eagle Medium-F12, Dulbecco's Modified Eagle Medium-Low Glucose, other basal medium or serum free medium or xeno free medium known in the art, fetal bovine serum, L-alanine-L-glutamine, penicillin and streptomycin or any combination thereof; and wherein the cell culture medium comprises basic fibroblast growth factor at a concentration of about 1 ng/mL to about 10 ng/mL, preferably about 2 ng/mL to enhance secretion of the bioactive factors.

13. (canceled)

14. The method as claimed in claim 1, wherein the conditioned medium obtained by fed batch activation is enriched for Ang-1; wherein the conditioned medium obtained by fed batch activation followed by complete medium change is enriched for TGF-b; and wherein the conditioned medium obtained by complete medium change is enriched for VEGF and PGE-2.

15. (canceled)

16. (canceled)

17. (canceled)

18. (canceled)

19. A conditioned medium comprising bioactive factors secreted by mesenchymal cells.

20. The conditioned medium as claimed in claim 19, wherein the bioactive factors comprises of biological active growth factors, cytokines, chemokines, anti-oxidants, small molecules, Extra cellular matrix (ECM) and other factors that are known to function or mediate a biological process, wherein the biological process includes cell proliferation and cell migration.

21. The conditioned medium, as claimed in claim 20, wherein the bioactive factors are selected from a group comprising VEGF, TGF-b, PGE-2, PDGF, GDNF, IGFBP, FGF, GCSF M-CSF angiogenin, angiopoietin, KGF, FGF7, BMP6, IGF1, laminin, MMP1, MMP2, MMP9, TIMP1, TIMP2, HGF, SDF1-LIF, IL-10 or any combination thereof; and wherein the conditioned medium, optionally contains amino acid selected from a group comprising L-Arginine and L-Glutamic acid or a combination thereof at a concentration ranging from about 50 mM to about 100 mM.

22. The conditioned medium as claimed in claim 21, wherein concentration of the VEGF ranges from about 2 to about 10 ng/mL; concentration of the TGF-b ranges from about 1 to about 5 ng/mL; concentration of the PGE-2 ranges from about 0.8 to about 2 ng/mL; concentration of the Angiopoietin-1 ranges from about 10 to about 12 ng/mL; concentration of the HGF ranges from about 20 to about 200 ng/ml; concentration of the SDF1 ranges from about 0.4 to about 3 ng/ml; and concentration of the IL-10 ranges from about 10 to about 50 ng/ml.

23.-28. (canceled)

29. The conditioned medium as claimed in claim 19, wherein the conditioned medium is present in a composition along with pharmaceutically acceptable excipient.

30. The conditioned medium as claimed in claim 29, wherein the excipient is selected from a group comprising additive, carrier, granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material and spheronization agents or any combination thereof.

31. The conditioned medium as claimed in claim 30, wherein the excipient is selected from a group comprising Hydroxy ethyl cellulose, Hydroxy propyl cellulose, Hydroxy propyl methyl cellulose, Carbopol, EDTA, Methyl paraben, Propyl paraben, Deionised water, Glycerin, DL-Panthenol, D-Panthenol, Phenoxyethanol, Allantoin, PEG, Purified water, Xanthan gum, Sodium PCA, Gluconolactone, Sodium Benzoate, Sodium Hydroxide, Phenoxyethanol, Ethylhexylglycerin, Sodium Polyacrylate, Caprylic or Capric Triglyceride, Mineral oil, Tri (PPG-3 Myristyl ether) Citrate, Sorbitan Laurate, Trideceth-6, PEG-75 Lanolin, Beta Glucan, Sodium Hyaluronate, Phosphatidylcholine, Cholesterol, Essential Oil, DL-α-Tocopherol acetate and Cyclodextrin or any combination thereof.

32. The conditioned medium as claimed in claim 29, wherein the composition is formulated into dosage forms selected from a group comprising, oily suspensions, hydrogel, nanogel, wet tissues, ointment, patch, gel, lotion, serum, emulsion, creams, spray, drops, or any combination thereof.

33. A method of managing skin related condition, said method comprising act of administering the conditioned medium of claim 19 or a formulation thereof, to a subject in need thereof.
